(19) United States
(12) Patent Application Publication
(10) Pub. No.: US 2026/0178970 A1
(43) Pub. Date: Jun. 25, 2026

(54) MAPPING SURGICAL WORKFLOWS INCLUDING MODEL MERGING

(71) Applicant: DIGITAL SURGERY LIMITED, London (GB)

(72) Inventors: Helena Elizabeth Anne JOHNSTON, London (GB); Carole RJ ADDIS, London (GB); Pinja ME HAIKKA, Hove (GB)

(21) Appl. No.: 19/127,248

(22) PCT Filed: Nov. 10, 2023
(86) PCT No.: PCT/EP2023/081517

§ 371 Date: May 5, 2025

Related U.S. Application Data
(60) Provisional application No. 63/424,218, filed on Nov. 10, 2022.

(51) Int. Cl. G06N 20/00

(52) U.S. Cl. CPC G06N 20/00

(57) ABSTRACT

An aspect includes model merging for surgical workflow mapping. Cases with one or more rare phases can be identified and removed. Remaining cases without the one or more rare phases can be clustered to form principal workflows. The principal workflows can be used to create a flow diagram for display via a user interface.

### BACKGROUND

The present disclosure relates in general to computing technology and relates more particularly to computing technology for model merging for surgical workflow mapping.

Computer-assisted systems, particularly computer-assisted surgery systems (CASs), rely on video data digitally captured during a surgery. Such video data can be stored and/or streamed. In some cases, the video data can be used to augment a person's physical sensing, perception, and reaction capabilities. For example, such systems can effectively provide the information corresponding to an expanded field of vision, both temporal and spatial, that enables a person to adjust current and future actions based on the part of an environment not included in his or her physical field of view. Alternatively, or in addition, the video data can be stored and/or transmitted for several purposes such as archival, training, post-surgery analysis, and/or patient consultation. The process of analyzing and comparing a large amount of video data from multiple surgical procedures to identify commonalities can be highly subjective and error-prone due, for example, to the volume of data and the numerous factors (e.g., patient condition, physician preferences, etc.) that impact the workflow of each individual surgical procedure that is being analyzed.

### SUMMARY

According to an aspect, a system includes a memory device and one or more processors coupled with the memory device. The one or more processors are configured to access a surgical dataset that includes a plurality of phases associated with workflows of a surgical procedure, identify and remove cases with one or more rare phases, and cluster remaining cases without the one or more rare phases to form principal workflows and identify outliers. The one or more processors are further configured to identify super-phases in the principal workflows, where the super-phases include subsequences of multiple phases. The one or more processors are further configured to create a flow diagram that masks the super-phases from the principal workflows.

According to another aspect, a computer-implemented method for extracting principal workflows from a set of surgical phase sequences includes receiving the set of surgical phase sequences; identifying rare phases in the set of surgical phase sequences; removing workflows containing the rare phases from the set of surgical phase sequences; clustering the workflows in the set of surgical phase sequences using iterative model-based clustering; generating a flow diagram based on the clustered surgical phase sequences; and/or outputting the flow diagram via a user interface.

According to a further aspect, a computer program product includes a memory device with computer readable instructions stored thereon, where executing the computer readable instructions by one or more processing units causes the one or more processing units to perform a plurality of operations. The operations include accessing a set of surgical phase sequences comprising a plurality of phases associated with workflows of a surgical procedure, identifying rare phases in the set of surgical phase sequences, removing workflows containing the rare phases from the set of surgical phase sequences, clustering the workflows in the set of surgical phase sequences, generating a flow diagram based on the clustered surgical phase sequences, and outputting the flow diagram via a user interface.

Additional technical features and benefits are realized through the techniques of the present invention. Aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 depicts a computer-assisted surgery (CAS) system according to one or more aspects;

FIG. 2 depicts a surgical procedure system according to one or more aspects;

FIG. 3 depicts a system for analyzing video captured by a video recording system according to one or more aspects;

FIG. 4 depicts a flowchart of a method for facilitating clinical interpretation of large and varied datasets of surgical workflow according to one or more aspects;

FIG. 5 depicts a process of an effective infrequent sequence miner (EIM);

FIG. 6 depicts a process of a modified EIM according to one or more aspects;

FIG. 7 depicts a visualization of a modified EIM according to one or more aspects;

FIG. 8 depicts transition matrices for EIM according to one or more aspects;

FIG. 9 depicts a process of a modified EIM according to one or more aspects;

FIG. 10 depicts a process of determining a similarity score and outlier identification according to one or more aspects;

FIG. 11 depicts a visualization of super-phase identification according to one or more aspects;

FIG. 12 depicts a visualization of key decision points in a surgical procedure according to one or more aspects;

FIG. 13 depicts a visualization of a transition matrix that identifies key points within the data where a decision is made according to one or more aspects;

FIG. 14 depicts a process of an alternative to EIM to create a clustered process model according to one or more aspects;

FIG. 15 depicts a hidden Markov model (HMM) according to one or more aspects;

FIG. 16 depicts Markov chain inputs according to one or more aspects;

FIG. 17 depicts Markov chain inputs according to one or more aspects;

FIG. 18 depicts a visualization of a workflow according to one or more aspects;

FIG. 19 depicts a visualization of a workflow according to one or more aspects;

FIG. 20 depicts an example of Bayesian Model Merging (BMM) according to one or more aspects;

FIG. 21 depicts a flow diagram of a method for creating a merged HMM according to one or more aspects;

FIG. 22 depicts a visualization of an example of a model having four sequences according to one or more aspects;

FIG. 23 depicts a merged model of the example of FIG. 22 according to one or more aspects;

FIG. 24 depicts a flow diagram of a method for creating a merged HMM according to one or more aspects;

FIG. 25 depicts a visualization of states based on workflows according to one or more aspects;

FIG. 26 depicts a visualization of states based on distinct workflows from the example of FIG. 25 according to one or more aspects;

FIG. 27 depicts pseudo code for a modified active trace clustering process according to one or more aspects;

FIG. 28 depicts process map visualizations of two clusters according to one or more aspects;

FIG. 29 depicts a process map visualization of combined clusters of FIG. 28 according to one or more aspects;

FIG. 30 depicts process map visualizations according to one or more aspects; and

FIG. 31 depicts a computer system according to one or more aspects.

The diagrams depicted herein are illustrative. There can be many variations to the diagrams and/or the operations described herein without departing from the spirit of the invention. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Aspects of the technical solutions described herein relate to analyzing surgical workflow data. Aspects include a process mining pipeline for extracting principal workflows from a set of surgical phase sequences. A dataset of surgical workflows with significant variation can be difficult to interpret. Aspects of the pipeline described herein identify groups of principal workflows and extract atypical workflows. A process map can be mined from the principal workflows for simpler visualization and clinical interpretation of the data.

In accordance with aspects, a method based on active trace clustering can be used to pick out homogenous groups within the dataset. This finds similar cases with common substrings and clusters them using a model-based approach. Atypical cases are identified and removed from the dataset, leaving the principal workflows. Principal workflows can be visualized using a modified Bayesian model merging (BMM) algorithm, which utilizes common substrings and allows multiple merges to increase computing efficiency.

Exemplary aspects of the technical solutions described herein include systems and methods for understanding the main workflows, identifying outliers, and understanding variation and key decision points in large datasets of surgical workflows. Aspects described herein can be used to facilitate clinical interpretation of large and varied dataset by identifying principal workflows, outliers, and decision points, and by pinpointing variation.

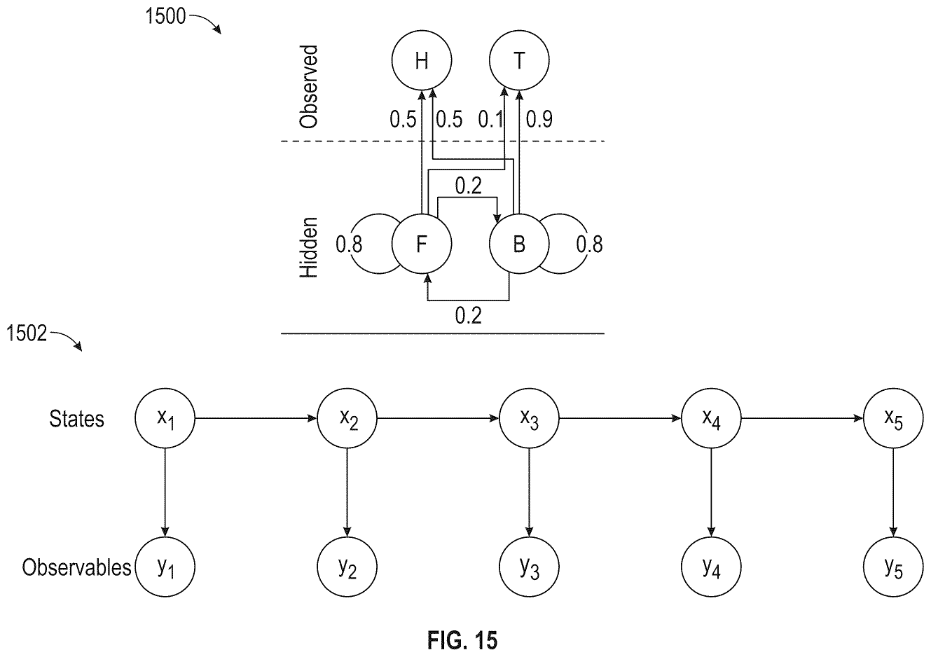

Aspects described herein can include the following steps: identify phases that rarely occur in any of the workflows; identify common sub-sequences, or substrings (super-phases) within the dataset and merge these into one phase; identify outlier workflows using an extension to an effective infrequent sequence mining method (EIM) (e.g., extension allows EIM to handle repeated phrases) or by clustering the workflows using an extension to Active Trace Clustering (“ATC” or “ActiTraC”) (e.g., extension includes cluster optimization to improve final clusters); remove outliers and compress sequences using the identified super-phases before carrying out BMM; and use BMM and/or phase transition matrices to identify decision points in the workflows. The decision points can be points in the model where there are multiple possible routes to take after a phase, or in a transition matrix where there are multiple phases that are likely transitions after a phase). The BMM aspects are described below in reference to FIGS. 15-26, for example.

Large-scale datasets of surgical workflows can be difficult to understand and interpret due to considerable variation in the way surgeries are performed. A workflow is the segmentation of a surgical procedure into a sequence of steps or phases; low-level information such as action-anatomy-instrument tuples may also be considered. As described herein, surgical cases can be broken into phases, the major events occurring in the surgery. Even high-level segmentation of a surgical case into phases can present prohibitive complexity for process modelling. In accordance with aspects described herein, a simplified representation for a large set of surgical workflows that will allow the building of a surgical process model is provided.

In according to aspects, process modeling has three main goals of discovering processes based on event logs, checking the conformance of new logs to a process model, and enhancing processes. Aspects described herein focus on process discovery, as it forms the basis for the latter two goals. Surgical process models can facilitate the clinical interpretation of large and varied datasets by compressing and visualizing the stages that constitute the surgical procedure. Visualization of surgical process models, in particular, is a challenging, yet valuable tool for the clinical interpretation of data. Process models also help with extracting outliers, identifying surgical decision points, and pinpointing variation in the dataset.

Aspects described herein include a pipeline for separating large surgical datasets into clusters of principal workflows and into a separate set of atypical workflows. A principal workflow is usually the most commonly occurring workflow, or a minor variation thereof. Major variations suggest the presence of alternative approaches and imply that the data should be split into smaller groups of low variation. In accordance with aspects, the dataset can be clustered using an extension of active trace clustering. This algorithm identifies similar cases based on common substrings and iteratively clusters the dataset based on the fitness and complexity of the resulting process map, using a heuristic miner. This results in clusters that generate representative and comprehensible process maps. Sequences that do not fit within any clusters or are highly unlikely given their cluster process model can be identified as atypical.

A surgical process may have sections that are near-deterministic and can be characterized by substrings of phases that follow a rigid order. Identifying these parts of the process can be interesting clinically and offers a twofold simplification for process modeling. A part of the clustering process can include creating a feature set to calculate similarity; a feature set used by aspects described herein utilizes common substrings within a dataset to create clusters with common substrings. Secondly, as a final preprocessing step, the common substrings are merged into “super-phases”, which according to one or more aspects, are made up of at least three consecutive phases. This provides compression of the dataset for the final process mapping.

In accordance with aspects, an extension of BMM can be used to create a process map to visualize each cluster. In accordance with aspects, restrictions on merging prevent any loops within the process map, resulting in an easily comprehensible map, even in the presence of repeated activities. In accordance with one or more aspects, additions have been made to this method to increase efficiency and identify an optimal number of merges based on the precision and complexity of the resulting map.

The pipeline described herein is procedure-agnostic and does not require expert input.

In exemplary aspects of the technical solutions described herein, surgical data that is captured by a computer-assisted surgical (CAS) system and segmented into surgical phases is input to the analysis described herein to to understand the main workflows, identify outliers and to understand the variation and key decision points.

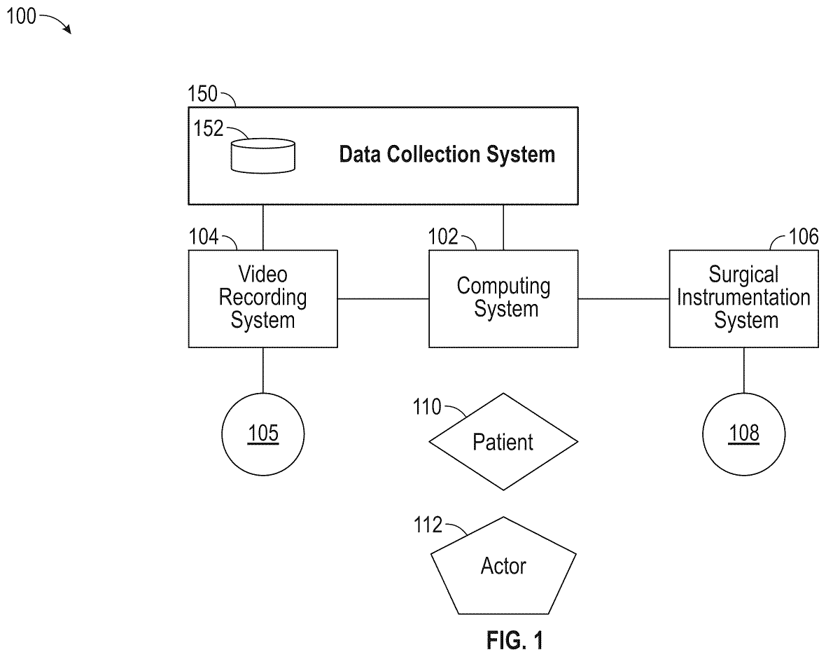

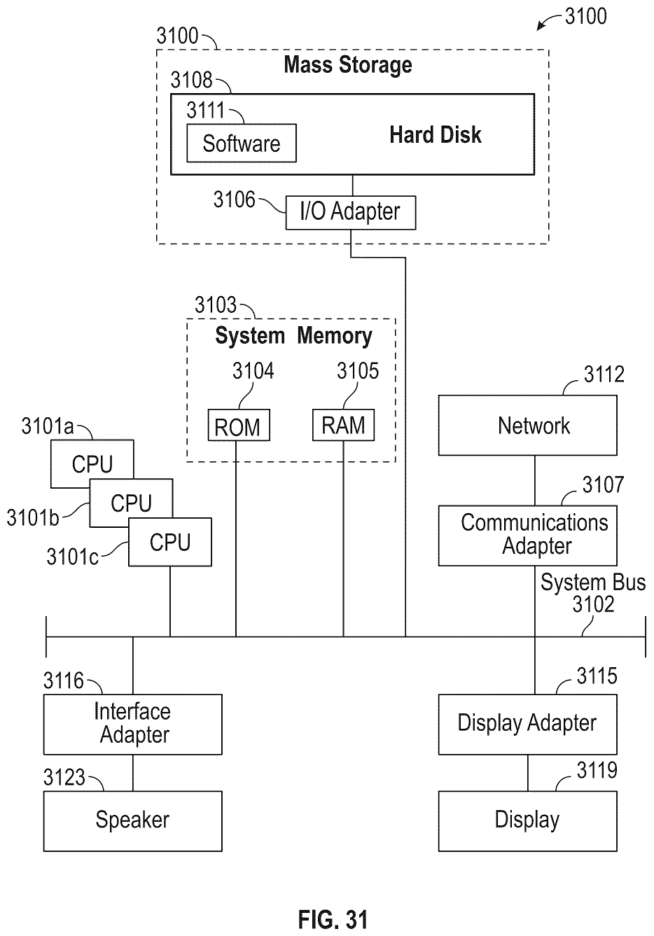

Turning now to FIG. 1, an example CAS system 100 is generally shown in accordance with one or more aspects. The CAS system 100 includes at least a computing system 102, a video recording system 104, and a surgical instrumentation system 106. All or a portion of the CAS system 100 can be implemented by all or a portion of computer system 3100 of FIG. 31 below. As illustrated in FIG. 1, an actor 112 can be medical personnel that uses the CAS system 100 to perform a surgical procedure on a patient 110. Medical personnel can be a surgeon, assistant, nurse, administrator, or any other actor that interacts with the CAS system 100 in a surgical environment. The surgical procedure can be any type of surgery, such as but not limited to cataract surgery, laparoscopic cholecystectomy, endoscopic endonasal transsphenoidal approach (eTSA) to resection of pituitary adenomas, or any other surgical procedure. In other examples, actor 112 can be a technician, an administrator, an engineer, or any other such personnel that interacts with the CAS system 100. For example, actor 112 can record data from the CAS system 100, configure/update one or more attributes of the CAS system 100, review past performance of the CAS system 100, repair the CAS system 100, etc.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g., closure). A “step” refers to the completion of a named surgical objective (e.g., hemostasis). During each step, certain surgical instruments 108 (e.g., forceps) are used to achieve a specific objective by performing one or more surgical actions. In addition, a particular anatomical structure of the patient may be the target of the surgical action(s).

The video recording system 104 includes one or more cameras 105, such as operating room cameras, endoscopic cameras, etc. The cameras 105 capture video data of the surgical procedure being performed. The video recording system 104 includes one or more video capture devices that can include cameras 105 placed in the surgical room to capture events surrounding (i.e., outside) the patient being operated upon. The video recording system 104 further includes cameras 105 that are passed inside (e.g., endoscopic cameras) the patient 110 to capture endoscopic data. The endoscopic data provides video and images of the surgical procedure.

The computing system 102 includes one or more memory devices, one or more processors, a user interface device, among other components. All or a portion of the computing system 102 shown in FIG. 1 can be implemented for example, by all or a portion of computer system 3100 of FIG. 31. Computing system 102 can execute one or more computer-executable instructions. The execution of the instructions facilitates the computing system 102 to perform one or more methods, including those described herein. The computing system 102 can communicate with other computing systems via a wired and/or a wireless network. In one or more examples, the computing system 102 includes one or more trained machine learning models that can detect and/or predict features of/from the surgical procedure that is being performed or has been performed earlier. Features can include structures such as anatomical structures, surgical instruments 108 in the captured video of the surgical procedure. Features can further include events such as phases, actions in the surgical procedure. Features that are detected can further include the actor 112 and/or patient 110. Based on the detection, the computing system 102, in one or more examples, can provide recommendations for subsequent actions to be taken by the actor 112. Alternatively, or in addition, the computing system 102 can provide one or more reports based on the detections. The detections by the machine learning models can be performed in an autonomous or semi-autonomous manner.

The machine learning models can include artificial neural networks, such as deep neural networks, convolutional neural networks, recurrent neural networks, encoders, decoders, or any other type of machine learning model. The machine learning models can be trained in a supervised, unsupervised, or hybrid manner. The machine learning models can be trained to perform detection and/or prediction using one or more types of data acquired by the CAS system 100. For example, the machine learning models can use the video data captured via the video recording system 104. Alternatively, or in addition, the machine learning models use the surgical instrumentation data from the surgical instrumentation system 106. In yet other examples, the machine learning models use a combination of video data and surgical instrumentation data.

Additionally, in some examples, the machine learning models can also use audio data captured during the surgical procedure. The audio data can include sounds emitted by the surgical instrumentation system 106 while activating one or more surgical instruments 108. Alternatively, or in addition, the audio data can include voice commands, snippets, or dialog from one or more actors 112. The audio data can further include sounds made by the surgical instruments 108 during their use.

In one or more examples, the machine learning models can detect surgical actions, surgical phases, anatomical structures, surgical instruments, and various other features from the data associated with a surgical procedure. The detection can be performed in real-time in some examples. Alternatively, or in addition, the computing system 102 analyzes the surgical data, i.e., the various types of data captured during the surgical procedure, in an offline manner (e.g., post-surgery). In one or more examples, the machine learning models detect surgical phases based on detecting some of the features such as the anatomical structure, surgical instruments, etc.

A data collection system 150 can be employed to store the surgical data, including the video(s) captured during the surgical procedures. The data collection system 150 includes one or more storage devices 152. The data collection system 150 can be a local storage system, a cloud-based storage system, or a combination thereof. Further, the data collection system 150 can use any type of cloud-based storage architecture, for example, public cloud, private cloud, hybrid cloud, etc. In some examples, the data collection system can use a distributed storage, i.e., the storage devices 152 are located at different geographic locations. The storage devices 152 can include any type of electronic data storage media used for recording machine-readable data, such as semiconductor-based, magnetic-based, optical-based storage media, or a combination thereof. For example, the data storage media can include flash-based solid-state drives (SSDs), magnetic-based hard disk drives, magnetic tape, optical discs, etc.

In one or more examples, the data collection system 150 can be part of the video recording system 104, or vice-versa. In some examples, the data collection system 150, the video recording system 104, and the computing system 102, can communicate with each other via a communication network, which can be wired, wireless, or a combination thereof. The communication between the systems can include the transfer of data (e.g., video data, instrumentation data, etc.), data manipulation commands (e.g., browse, copy, paste, move, delete, create, compress, etc.), data manipulation results, etc. In one or more examples, the computing system 102 can manipulate the data already stored/being stored in the data collection system 150 based on outputs from the one or more machine learning models, e.g., phase detection, anatomical structure detection, surgical tool detection, etc. Alternatively, or in addition, the computing system 102 can manipulate the data already stored/being stored in the data collection system 150 based on information from the surgical instrumentation system 106.

In one or more examples, the video captured by the video recording system 104 is stored on the data collection system 150. In some examples, the computing system 102 curates parts of the video data being stored on the data collection system 150. In some examples, the computing system 102 filters the video captured by the video recording system 104 before it is stored on the data collection system 150. Alternatively, or in addition, the computing system 102 filters the video captured by the video recording system 104 after it is stored on the data collection system 150.

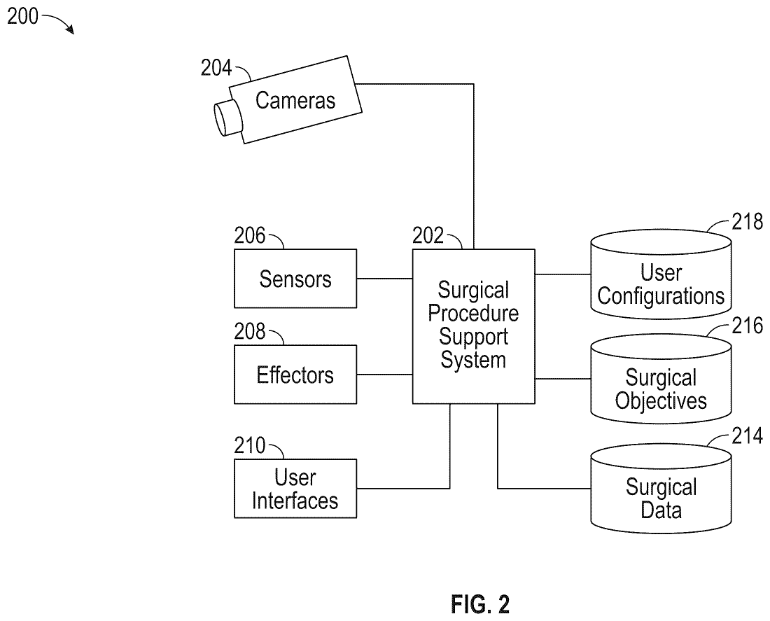

Turning now to FIG. 2, a surgical procedure system 200 is generally shown in accordance with one or more aspects. The example of FIG. 2 depicts a surgical procedure support system 202 that can include or may be coupled to the CAS system 100 of FIG. 1. The surgical procedure support system 202 can acquire image or video data using one or more cameras 204. The surgical procedure support system 202 can also interface with a plurality of sensors 206 and effectors 208. The sensors 206 may be associated with surgical support equipment and/or patient monitoring. The effectors 208 can be robotic components or other equipment controllable through the surgical procedure support system 202. The surgical procedure support system 202 can also interact with one or more user interfaces 210, such as various input and/or output devices. The surgical procedure support system 202 can store, access, and/or update surgical data 214 associated with a training dataset and/or live data as a surgical procedure is being performed on patient 110 of FIG. 1. The surgical procedure support system 202 can store, access, and/or update surgical objectives 216 to assist in training and guidance for one or more surgical procedures. User configurations 218 can track and store user preferences.

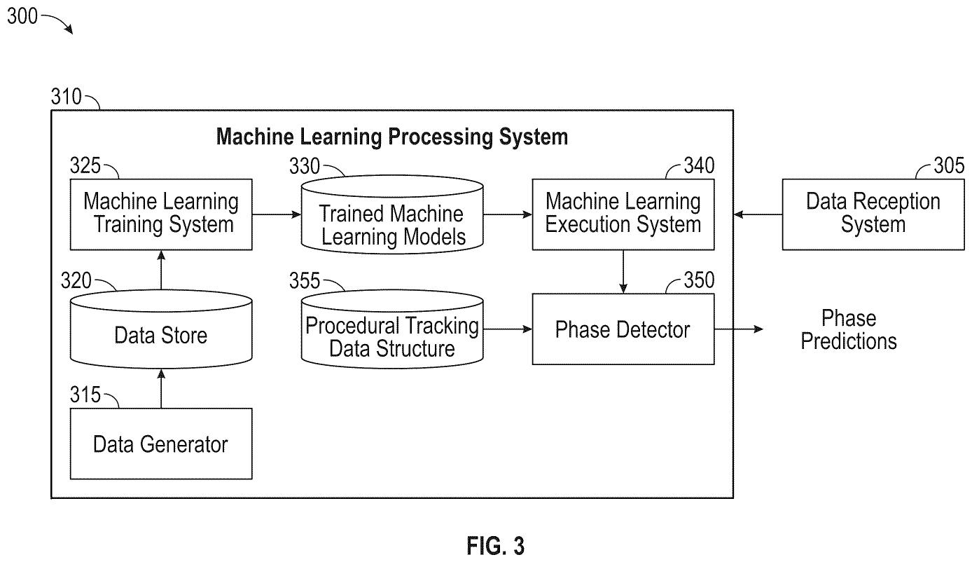

Turning now to FIG. 3, a system 300 for analyzing video and data is generally shown according to one or more aspects. In accordance with aspects, the video and data is captured from video recording system 104 of FIG. 1. The analysis can result in predicting features that include surgical phases and structures (e.g., instruments, anatomical structures, etc.) in the video data using machine learning. System 300 can be the computing system 102 of FIG. 1, or a part thereof in one or more examples. System 300 uses data streams in the surgical data to identify procedural states according to some aspects.

System 300 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed. Alternatively, or in addition, the data reception system 305 can receive or access surgical data in an offline manner, for example, by accessing data that is stored in the data collection system 150 of FIG. 1.

System 300 further includes a machine learning processing system 310 that processes the surgical data using one or more machine learning models to identify one or more features, such as surgical phase, instrument, anatomical structure, etc., in the surgical data. It will be appreciated that machine learning processing system 310 can include one or more devices (e.g., one or more servers), each of which can be configured to include part or all of one or more of the depicted components of the machine learning processing system 310. In some instances, a part or all of the machine learning processing system 310 is in the cloud and/or remote from an operating room and/or physical location corresponding to a part or all of data reception system 305. It will be appreciated that several components of the machine learning processing system 310 are depicted and described herein. However, the components are just one example structure of the machine learning processing system 310, and that in other examples, the machine learning processing system 310 can be structured using a different combination of the components. Such variations in the combination of the components are encompassed by the technical solutions described herein.

The machine learning processing system 310 includes a machine learning training system 325, which can be a separate device (e.g., server) that stores its output as one or more trained machine learning models 330. The machine learning models 330 are accessible by a machine learning execution system 340. The machine learning execution system 340 can be separate from the machine learning training system 325 in some examples. In other words, in some aspects, devices that “train” the models are separate from devices that “infer,” i.e., perform real-time processing of surgical data using the trained machine learning models 330.

Machine learning processing system 310, in some examples, further includes a data generator 315 to generate simulated surgical data, such as a set of virtual images, or record the video data from the video recording system 104, to train the machine learning models 330. Data generator 315 can access (read/write) a data store 320 to record data, including multiple images and/or multiple videos. The images and/or videos can include images and/or videos collected during one or more procedures (e.g., one or more surgical procedures). For example, the images and/or video may have been collected by a user device worn by the actor 112 of FIG. 1 (e.g., surgeon, surgical nurse, anesthesiologist, etc.) during the surgery, a non-wearable imaging device located within an operating room, or an endoscopic camera inserted inside the patient 110 of FIG. 1. The data store 320 is separate from the data collection system 150 of FIG. 1 in some examples. In other examples, the data store 320 is part of the data collection system 150.

Each of the images and/or videos recorded in the data store 320 for training the machine learning models 330 can be defined as a base image and can be associated with other data that characterizes an associated procedure and/or rendering specifications. For example, the other data can identify a type of procedure, a location of a procedure, one or more people involved in performing the procedure, surgical objectives, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, etc.). Further, the other data can include image-segmentation data that identifies and/or characterizes one or more objects (e.g., tools, anatomical objects, etc.) that are depicted in the image or video. The characterization can indicate the position, orientation, or pose of the object in the image. For example, the characterization can indicate a set of pixels that correspond to the object and/or a state of the object resulting from a past or current user handling. Localization can be performed using a variety of techniques for identifying objects in one or more coordinate systems.

The machine learning training system 325 uses the recorded data in the data store 320, which can include the simulated surgical data (e.g., set of virtual images) and actual surgical data to train the machine learning models 330. The machine learning model 330 can be defined based on a type of model and a set of hyperparameters (e.g., defined based on input from a client device). The machine learning models 330 can be configured based on a set of parameters that can be dynamically defined based on (e.g., continuous or repeated) training (i.e., learning, parameter tuning). Machine learning training system 325 can use one or more optimization algorithms to define the set of parameters to minimize or maximize one or more loss functions. The set of (learned) parameters can be stored as part of a trained machine learning model 330 using a specific data structure for that trained machine learning model 330. The data structure can also include one or more non-learnable variables (e.g., hyperparameters and/or model definitions).

Machine learning execution system 340 can access the data structure(s) of the machine learning models 330 and accordingly configure the machine learning models 330 for inference (i.e., prediction). The machine learning models 330 can include, for example, a fully convolutional network adaptation, an adversarial network model, an encoder, a decoder, or other types of machine learning models. The type of the machine learning models 330 can be indicated in the corresponding data structures. The machine learning model 330 can be configured in accordance with one or more hyperparameters and the set of learned parameters.

The machine learning models 330, during execution, receive, as input, surgical data to be processed and subsequently generate one or more inferences according to the training. For example, the video data captured by the video recording system 104 of FIG. 1 can include data streams (e.g., an array of intensity, depth, and/or RGB values) for a single image or for each of a set of frames (e.g., including multiple images or an image with sequencing data) representing a temporal window of fixed or variable length in a video. The video data that is captured by the video recording system 104 can be received by the data reception system 305, which can include one or more devices located within an operating room where the surgical procedure is being performed. Alternatively, the data reception system 305 can include devices that are located remotely, to which the captured video data is streamed live during the performance of the surgical procedure. Alternatively, or in addition, the data reception system 305 accesses the data in an offline manner from the data collection system 150 or from any other data source (e.g., local or remote storage device).

The data reception system 305 can process the video and/or data received. The processing can include decoding when a video stream is received in an encoded format such that data for a sequence of images can be extracted and processed. The data reception system 305 can also process other types of data included in the input surgical data. For example, the surgical data can include additional data streams, such as audio data, RFID data, textual data, measurements from one or more surgical instruments/sensors, etc., that can represent stimuli/procedural states from the operating room. The data reception system 305 synchronizes the different inputs from the different devices/sensors before inputting them in the machine learning processing system 310.

The machine learning models 330, once trained, can analyze the input surgical data, and in one or more aspects, predict and/or characterize features (e.g., structures) included in the video data included with the surgical data. The video data can include sequential images and/or encoded video data (e.g., using digital video file/stream formats and/or codecs, such as MP4, MOV, AVI, WEBM, AVCHD, OGG, etc.). The prediction and/or characterization of the features can include segmenting the video data or predicting the localization of the structures with a probabilistic heatmap. In some instances, the one or more machine learning models include or are associated with a preprocessing or augmentation (e.g., intensity normalization, resizing, cropping, etc.) that is performed prior to segmenting the video data. An output of the one or more machine learning models can include image-segmentation or probabilistic heatmap data that indicates which (if any) of a defined set of structures are predicted within the video data, a location and/or position and/or pose of the structure(s) within the video data, and/or state of the structure(s). The location can be a set of coordinates in an image/frame in the video data. For example, the coordinates can provide a bounding box. The coordinates can provide boundaries that surround the structure(s) being predicted. The machine learning models 330, in one or more examples, are trained to perform higher-level predictions and tracking, such as predicting a phase of a surgical procedure and tracking one or more surgical instruments used in the surgical procedure.

While some techniques for predicting a surgical phase (“phase”) in the surgical procedure are described herein, it should be understood that any other technique for phase prediction can be used without affecting the aspects of the technical solutions described herein. In some examples, the machine learning processing system 310 includes a phase detector 350 that uses the machine learning models to identify a phase within the surgical procedure (“procedure”). Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by actor 112. The procedural tracking data structure 355 identifies a set of potential phases that can correspond to a part of the specific type of procedure.

In some examples, the procedural tracking data structure 355 can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure 355 may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state can indicate a complication (e.g., blood clots, clogged arteries/veins, etc.), pre-condition (e.g., lesions, polyps, etc.). In some examples, the machine learning models 330 are trained to detect an “abnormal condition,” such as hemorrhaging, arrhythmias, blood vessel abnormality, etc.

Each node within the procedural tracking data structure 355 can identify one or more characteristics of the phase corresponding to that node. The characteristics can include visual characteristics. In some instances, the node identifies one or more tools that are typically in use or availed for use (e.g., on a tool tray) during the phase. The node also identifies one or more roles of people who are typically performing a surgical task, a typical type of movement (e.g., of a hand or tool), etc. Thus, phase detector 350 can use the segmented data generated by machine learning execution system 340 that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, etc.).

The phase detector 350 outputs the phase prediction associated with a portion of the video data that is analyzed by the machine learning processing system 310. The phase prediction is associated with the portion of the video data by identifying a start time and an end time of the portion of the video that is analyzed by the machine learning execution system 340. The phase prediction that is output can include segments of the video where each segment corresponds to and includes an identity of a surgical phase as detected by the phase detector 350 based on the output of the machine learning execution system 340. Further, the phase prediction, in one or more examples, can include additional data dimensions such as, but not limited to, identities of the structures (e.g., instrument, anatomy, etc.) that are identified by the machine learning execution system 340 in the portion of the video that is analyzed. The phase prediction can also include a confidence score of the prediction. Other examples can include various other types of information in the phase prediction that is output.

It should be noted that although some of the drawings depict endoscopic videos being analyzed, the technical solutions described herein can be applied to analyze video and image data captured by cameras that are not endoscopic (i.e., cameras external to the patient's body) when performing open surgeries (i.e., not laparoscopic surgeries). For example, the video and image data can be captured by cameras that are mounted on one or more personnel in the operating room, e.g., surgeon. Alternatively, or in addition, the cameras can be mounted on surgical instruments, walls, or other locations in the operating room.

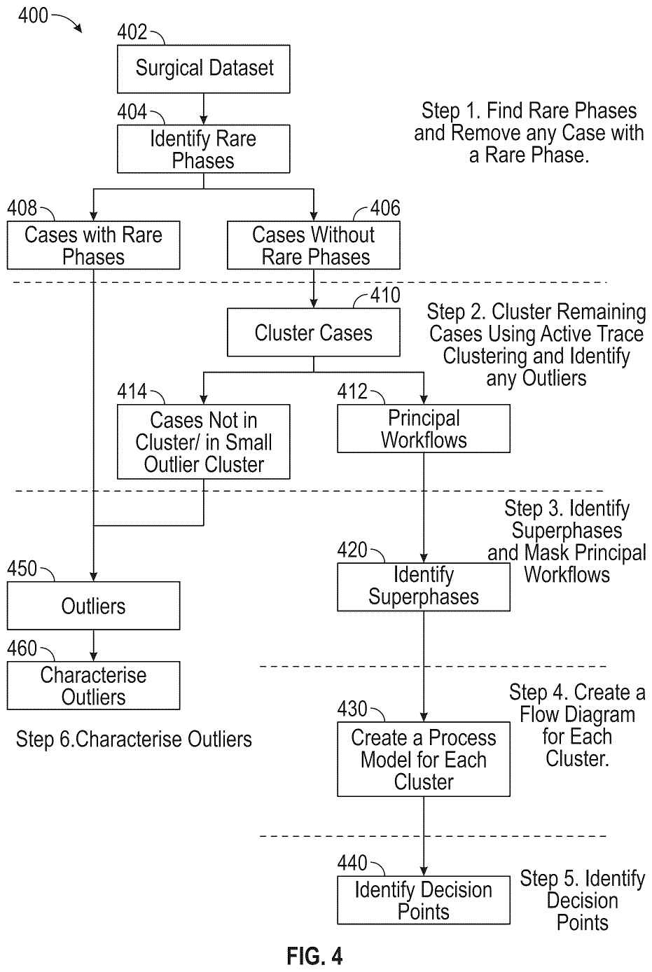

Turning now to FIG. 4, a flowchart of a method 400 for facilitating clinical interpretation of large and varied datasets of surgical workflow is generally shown in accordance with one or more aspects. All or a portion of the processing shown in the method 400 of FIG. 4 can be performed for example, by computing system 102 of FIG. 1, or a part thereof in one or more examples. The method 400 shown in FIG. 4 includes using super-phases as a way of simplifying sequences, using extensions to BMM, using extensions to EIM (e.g., use of super-phases and higher order transition matrices), and combining BMM and EIM.

The method 400 shown in FIG. 4 is a pipeline of different methods including: identifying rare phases and transitions; clustering of sequences and outlier identification using a modified version of Active Trace Clustering; super-phase identification by finding common subsequences to compress the workflows; a modified version of BMM to create a flow diagram of the data; and identification of decision points using BMM or transition matrices. Aspects related to using the modified version of BMM to create a flow diagram of the data and identification of decision points using BMM are described further herein in reference to FIGS. 15-26.

In a first step of method 400, a surgical dataset 402 is accessed, rare phases 404 are identified and partitioned as cases without rare phases 406 and cases with rare phases 408. In a second step of method 400, the cases without rare phases 406 are clustered 410 with principal workflows 412 identified and cases not clustered 414 or in small outlier clusters identified. In a third step of method 400, super-phases 420 are identified from the principal workflows 412. In a fourth step of method 400, a process model 430 or flow diagram can be created from each cluster of the principal workflows 412. In a fifth step of the method 400, decision points 440 can be identified in the process model 430. The cases not clustered 414 or in small outlier clusters can be identified as outliers 450 and the outliers can be characterized 460 in a sixth step of the method 400.

The processing shown in FIG. 4 is not intended to indicate that the operations are to be executed in any particular order or that all of the operations shown in FIG. 4 are to be included in every case. Additionally, the processing shown in FIG. 4 can include any suitable number of additional operations.

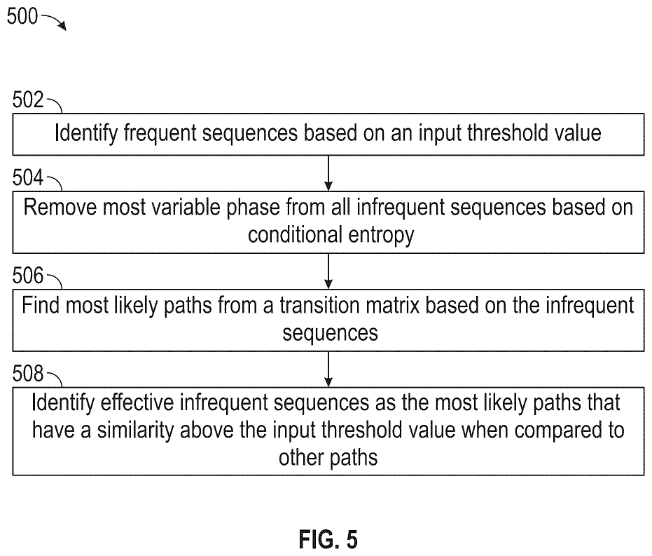

Turning now to FIG. 5, a process 500 performed by an EIM is generally shown. The EIM can be used to discover infrequent sequences in sequences of data that represent important variations within the data. These infrequent sequences that represent important variations within the data should not be discarded, but should be kept and used by the modeling of the data. At block 502, frequent sequences are identified based on an input threshold value. At block 504, the most variable phase based on conditional entropy is removed from all infrequent sequences (e.g., remove the noise in the system). If there is no noise within the data, phases need not be removed. At block 506, the most likely paths are found from a transition matrix based on the infrequent sequences. At block 508, the most likely paths that have a similarity above the input threshold value, when compared to the other paths, are identified as the effective infrequent sequences.

The processing shown in FIG. 5 is not intended to indicate that the operations are to be executed in any particular order or that all of the operations shown in FIG. 5 are to be included in every case. Additionally, the processing shown in FIG. 5 can include any suitable number of additional operations.

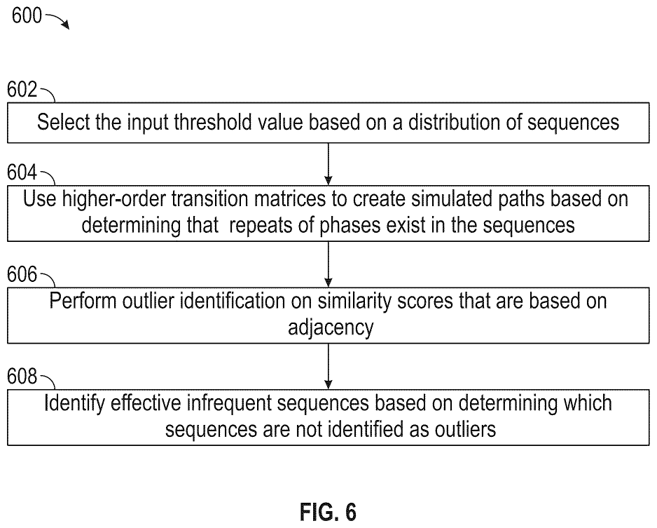

Turning now to FIG. 6, a process 600 performed by a modified EIM is generally shown in accordance with one or more aspects. The processing of the modified EIM shown in FIG. 6 can account for characteristics of surgical workflow data including for example, the possibility of repeated phases in a workflow sequence (or sequence of phases) and remove the need for manual setting of the frequent threshold and similarity threshold, replacing it with an automated approach. This method could be used within the pipeline, in addition to or instead of the active trace clustering, to identify outlier sequences. At block 602, the input threshold value of block 502 can be selected based on a distribution of sequences. At block 604, higher-order transition matrices can be used to create simulated paths based on determining that repeats of phases exist in the sequences. This can be performed in place of block 506. At block 606, outlier identification can be performed on similarity scores that are based on adjacency. At block 608, effective infrequent sequences can be identified based on determining which sequences are not identified as outliers. Blocks 606 and 608 can be performed as an alternative to finding paths that are above the input threshold value in block 508.

The processing shown in FIG. 6 is not intended to indicate that the operations are to be executed in any particular order or that all of the operations shown in FIG. 6 are to be included in every case. Additionally, the processing shown in FIG. 6 can include any suitable number of additional operations.

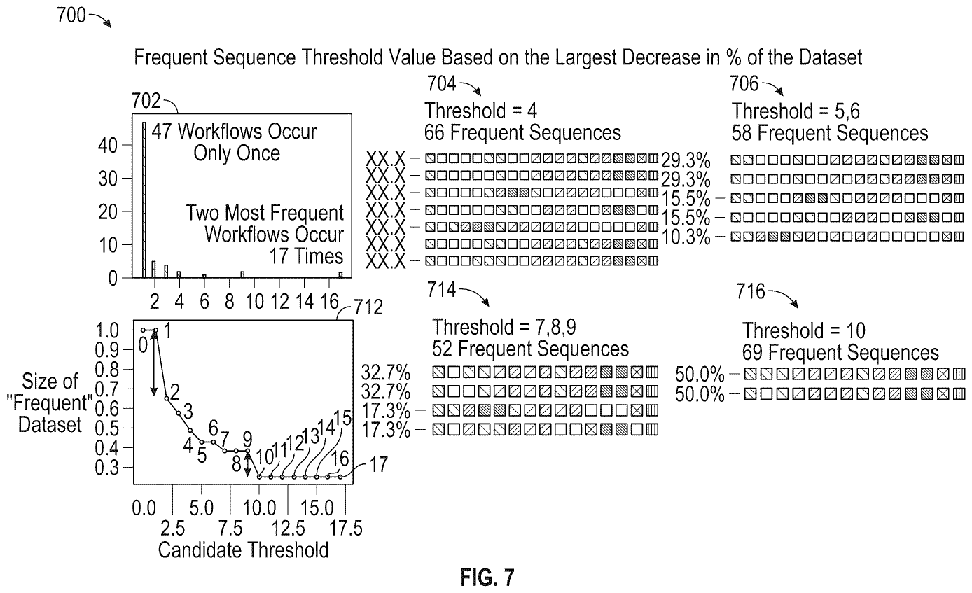

Turning now to FIG. 7, a visualization 700 of the modified EIM is generally shown in accordance with one or more aspects. As shown in FIG. 7, a threshold value can be selected based on the threshold value that results in the largest decrease in percentage of the dataset. In the top graph 702, the vertical scale represents a number of workflows and the horizontal scale represents a number of times of occurrence. For example, as shown in the top graph 702, 47 workflows occur only once. An example is depicted in chart 704 for 66 frequent sequences and a threshold of 4. Chart 706 depicts an example of 58 frequent sequences with thresholds of 5 and 6. An alternate method of selecting the frequency sequence threshold value is shown in the bottom graph 712, where the biggest drops in the size of the dataset of “frequent” sequences is used to select the threshold. Using this approach, workflows that are before the biggest drop are considered to be frequent and workflows that are after the drop are considered to be infrequent. This method can be automated to replace the need for a manually set frequency threshold. Further example charts 714 and 716 are depicted, where chart 714 depicts 52 frequent sequences with thresholds of 7, 8, 9, and chart 716 depicts 69 frequent sequences with a threshold of 10.

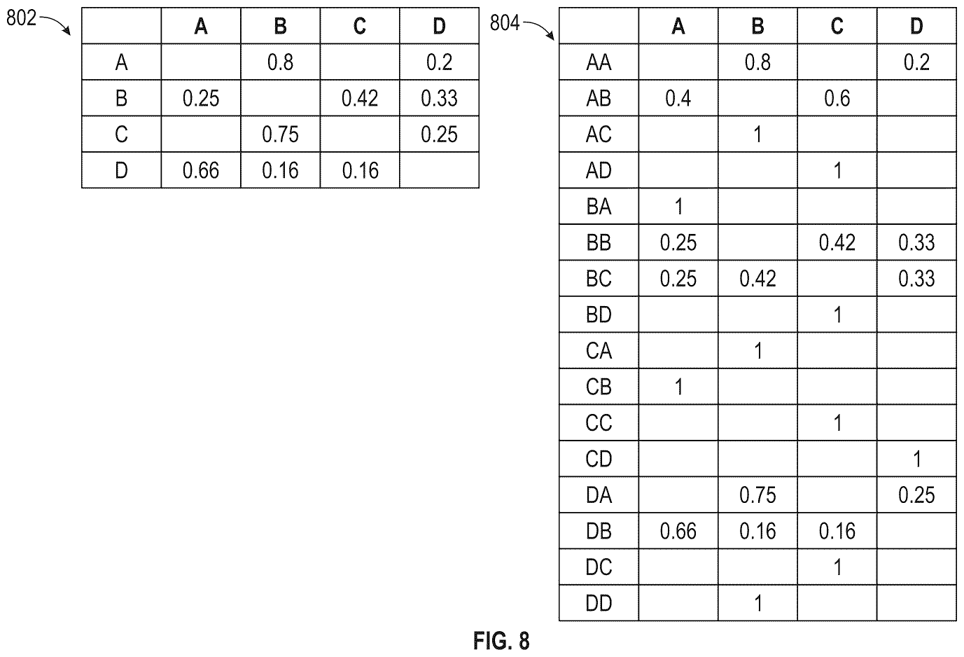

Turning now to FIG. 8, transition matrices 802, 804 for an EIM extension are generally shown in accordance with one or more aspects. The most likely paths are found from a transition matrix based on the infrequent sequences. When there are repeated phases in the data the most likely path within the transition matrix may get caught in an infinite loop (e.g., if most likely to go from phase A to phase B, and most likely to go from phase B to phase A, the most likely path will be an infinite loop between A and B). To account for this higher order transition matrices can be used where applicable-these include phases before the current phase in the transition matrix.

The matrix 802 to the left in FIG. 8 shows the most likely path from A as: A->B->C->B->C . . . etc. (i.e., can never return to A).

The matrix 804 to the right in FIG. 8 shows a second order transition matrix (an example of a higher order transition matrix) that looks at the likelihood of the next phase based on the two previous phases. The most likely path from A, B as shown in the second order matrix is: A->B->C->B->A. By using a higher order transition matrix, the method's ability to identify outliers can be improved on data with repeated phases and toggling.

The use of super-phases within this method may also improve the ability to deal with repeated phases and toggling. For example, if B->C->B is a common subsequence this may become a new super-phase “BCB” which is included as a single entry in the matrix 802 on the left.

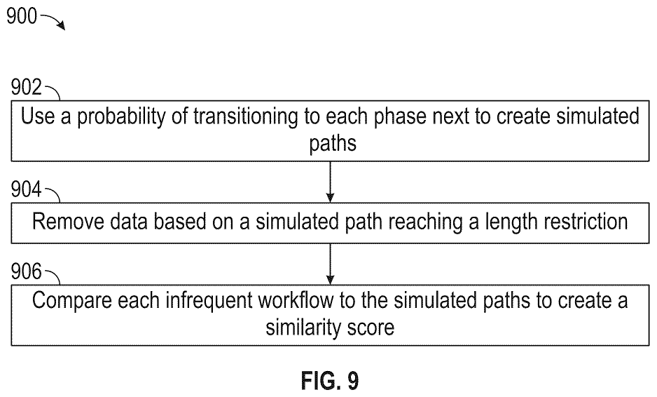

Turning now to FIG. 9, a process 900 of a modified EIM, where a transition matrix is used to create simulated data, is generally shown in accordance with one or more aspect. This approach can be used to remove any biases towards longer sequences and to reduce loops. At block 902, a probability of transitioning to each phase next can be used to create simulated paths. This can be used rather than finding the most likely paths. At block 904, data based on a simulated path reaching a length restriction can be removed. At block 906, each infrequent workflow can be compared to the simulated paths to create a similarity score.

The processing shown in FIG. 9 is not intended to indicate that the operations are to be executed in any particular order or that all of the operations shown in FIG. 9 are to be included in every case. Additionally, the processing shown in FIG. 9 can include any suitable number of additional operations.

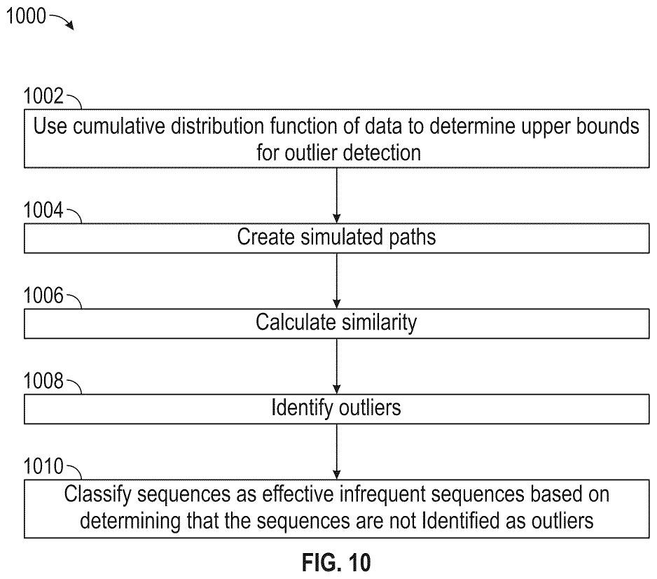

Turning now to FIG. 10, a process 1000 of calculating a similarity score and outlier identification using a modified EIM is generally shown in accordance with one or more aspects. In some aspects, rather than using an edit distance, the adjacency score will be used. A benefit of using the adjacency score, is that this is based on the transitions in both sequences. The edit distance is the cost to edit one sequence to make it into another, so if one sequence is much longer than the other the edit distance will always be high. When comparing with the adjacency score, the score will be high if there are transitions present in one that is not in the other.

For outlier identification, at block 1002, cumulative distribution function of data can be used to determine upper bounds for outlier detection. This approach can be more reliable than manually setting a threshold. At block 1004, simulated paths can be created. At block 1006, similarity can be calculated. At block 1008, outliers can be identified. At block 1010, sequences can be classified as effective infrequent sequences based on determining that the sequences are not identified as outliers.

The processing shown in FIG. 10 is not intended to indicate that the operations are to be executed in any particular order or that all of the operations shown in FIG. 10 are to be included in every case. Additionally, the processing shown in FIG. 10 can include any suitable number of additional operations.

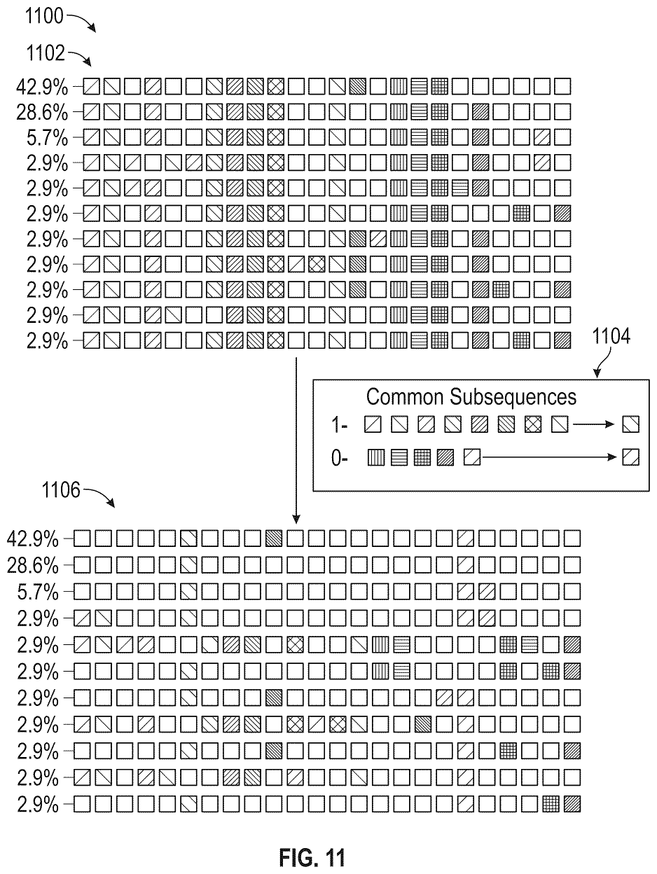

Turning now to FIG. 11, a visualization 1100 of super-phase identification is generally shown in accordance with one or more aspects. As shown in FIG. 11, the principal workflows are compressed by creating super-phases. Super-phases include subsequences of a specified length (e.g., two, three, four, five, etc.) and that appear commonly within the data. With respect to workflows of chart 1102, two super-phases, referred to as “common subsequences” 1104 are shown in FIG. 11. Super-phases are deterministic sequences of phases, meaning that they can be used to simplify the data before BMM, making the merging process much easier because there is less data to handle. As shown in the bottom chart 1106 of FIG. 11, the two super-phases are substituted into the workflow sequence, resulting in simplified data. The use of super-phases may improve the ability to deal with repeated phases and toggling. For example, if B->C->B is a common subsequence in chart 1102 this may become a new super-phase “BCB” which would be included as a single entry in the chart 1106.

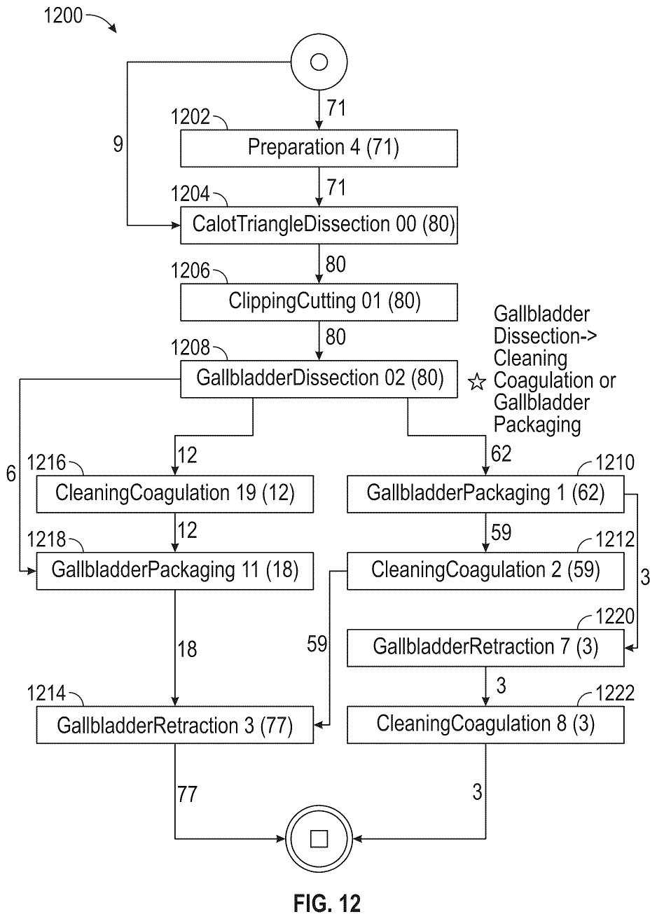

Turning now to FIG. 12, a visualization 1200 of key decision points in a surgical procedure is generally shown in accordance with one or more aspects. In the example shown in FIG. 12, eighty workflows are analyzed with the “Preparation 4” phase being the first phase 71 in block 1202 of the workflows and the “CalotTriangleDissection 00” phase in block 1204 being the first phase in 9 of the workflows. The shaded path is the most likely path through the surgery (blocks 1206, 1208, 1210, 1212, and 1214) and the star represents a decision point at block 1208 which occurs after the gallbladder dissection phase (e.g., to proceed to the cleaning coagulation phase or to the gallbladder packaging phase). Other paths include blocks 1216, 1218 branching from block 1208 as possible paths before rejoining block 124. A further alternate path branches from block 1210 and includes blocks 1220 and 1222.

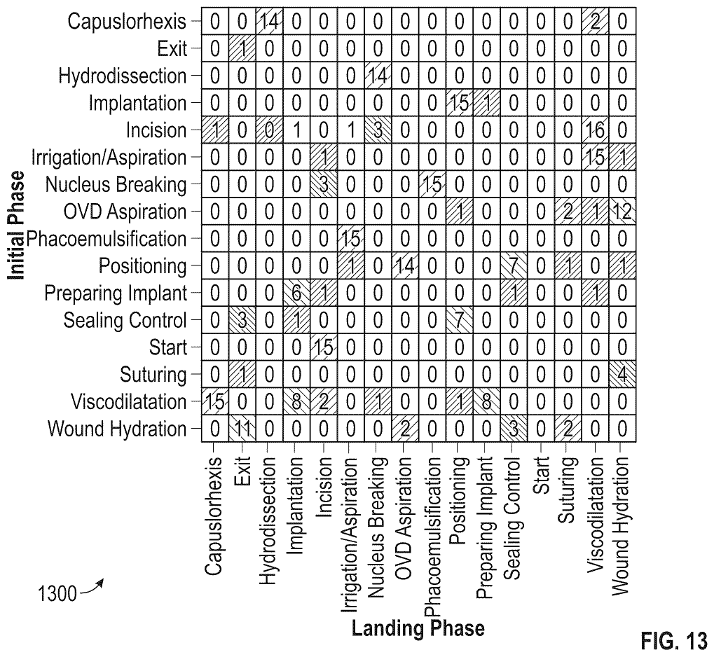

Turing now to FIG. 13, a visualization 1300 of a transition matrix that identifies key points within the data where a decision is made is generally shown in accordance with one or more aspects. In the example shown, a decision point would be at the Viscodilatation phase, where there are three likely following phases of Capsulorhexis, Implantation and Preparing Implant.

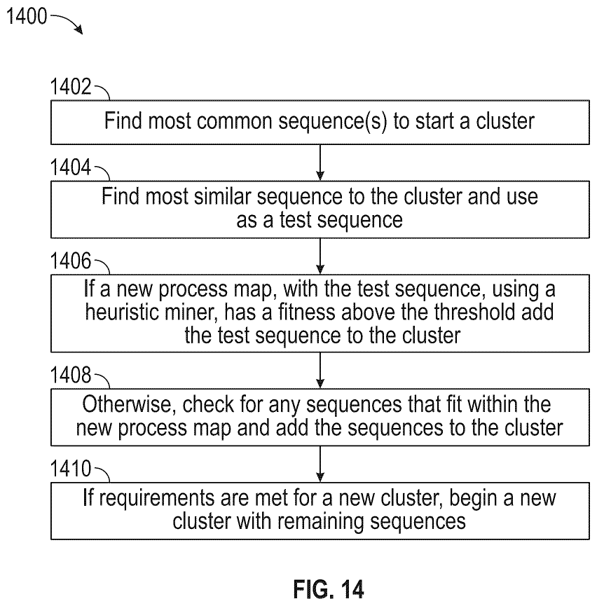

Turning now to FIG. 14, a process 1400 of an alternative to Active trace clustering to create a clustered process model is generally shown in accordance with one or more aspects. The process shown in FIG. 14 can be referred to as a type of model-based trace clustering. At block 1402, searching can be performed to find most common sequence(s) to start a cluster. At block 1404, searching can be performed to find a most similar sequence to the cluster and use as a test sequence. At block 1406, if a new process map, with the test sequence, using a heuristic miner, has a fitness above the threshold the test sequence is added to the cluster. At block 1408, otherwise, a check can be performed for any sequences that fit within the new process map and the sequences can be added to the cluster. At block 1410, if requirements are met for a new cluster, a new cluster can begin with remaining sequences.

In some aspects, various modification can include using a vector based feature set and Euclidean distance where similarity between sequences was calculated based on common substrings within a cluster. Further, an additional constraint can be added for a test sequence to be added to a cluster. A maximum distance between sequences in a cluster below threshold can be used as a constraint, for example. This can ensure that each cluster has a restricted amount of possible variation. An additional step can be added to optimize clusters based on process model likelihoods from each cluster. This can ensure each sequence is a best possible cluster after convergence. This is carried out by finding the likelihood of each sequence in each cluster Heuristic Miner model and placing it in the cluster with the highest likelihood. A new cluster can be started if, for instance, the number of remaining sequences is more than a minimum cluster size.

The processing shown in FIG. 14 is not intended to indicate that the operations are to be executed in any particular order or that all of the operations shown in FIG. 14 are to be included in every case. Additionally, the processing shown in FIG. 14 can include any suitable number of additional operations.

In according to an aspect, the modified active trace clustering can include using model-based clustering methods and the following pipeline: identify rare phases; cluster the remaining sequences using active trace clustering (using distance measurements based on common subsequences to identify which sequence should be tested in the cluster next; clustering sequences based on the resulting Heuristic Miner model; and after clustering running optimization on the clusters by finding which clusters in each sequence are most likely to be in based on the cluster process models and repeating this process until the clusters remain constant to ensure the best possible clustering has been identified); compress the clusters based on the super-phases to simplify the data; run the model merging algorithm on each cluster to get the process model; and identify outliers as any sequences not in a cluster or in a very small cluster (e.g., less than five sequences or less than ten sequences) or as those that are outliers based on the process model likelihoods. This approach may have advantages when the dataset is a complex dataset.

Turning now to FIGS. 15-26 , model merging for a surgical workflow map is generally shown and described in according to one or more aspects. Aspects of the model merging can be used to perform Bayesian model merging, which can include visualization of principal workflows and identification of surgical decision points. Input can include masked principal workflows as described previously herein. The use of a model to visualize a user's workflows can be a good way to show the user how their workflow varies in an intuitive flow chart. Also embedded within the user's procedure map can be useful information including clinically relevant insights and key decision points within their procedure map.

Models can be used to map out surgical processes to give a flow chart like model. Contemporary approaches have included using a model merging process, which can be configured to not allow loops, meaning that the model can read from left to right (or top to bottom) making a clear and easy to understand flow chart. The use of a visualization like this is a good way to show users their different workflows for a procedure. It may also allow incorporation of information from the surgical maps to include clinical information within the visualization. This allows the extraction of this clinical information more easily and quickly.

The model merging can be used to create a model of surgical data, with the states within the model being iteratively merged to improve the score of the model based on the input observations. It works by maximizing the probability of the model, given the training data. For a model M and data D, the P(M|D) is proportional to P(D|M)P(M). P(M) can be found using the forward algorithm or an approximate value using Viterbi paths. Merges of states are allowed if they give the same observation and if they maximize P(D|M). At each iteration of the process two states are merged together to simplify the model. The two states to be merged are chosen on the below criteria; they result in the same observation (phase), the merge results in the highest P(D|M) for the data out of all the possible merges, and the merge doesn't result in a loop in the model (added to ensure the models are easy to read from left to right). The model continues merging states until there are no more possible merges or until the maximum number of iterations has been reached or until the score of the model has converged.

Aspects described herein extend the current methodology used for the merging process to make it more robust and efficient when looking at surgical phase data. At the start of the modeling process each observation is treated as unique and all its values are given a unique state value. However, for surgical workflow data there are often multiple observations which contain the same set of values. To improve the speed of the model merging process the same observations are collected together and given the same state values. This is the same as essentially merging all the states that are part of the same observation workflow together before continuing the merging process. This can greatly reduce the number of states in the model at the start, thereby speeding up the merging process significantly.

At each iteration in the current methodology only one merge is applied to the model. In according to aspects described herein multiple merges are allowed, which can speed up the merging process. Multiple merges are only allowed given the following conditions: all the merges have the highest P(D|M); and none of the merges will interact with each other (i.e., every route through the model does not pass through more than one merge).

The current method prevents any merges that would result in loops in the model to ensure that the model is able to be read from left to right. In one or more aspects described herein an additional requirement is added to specify that a merge cannot result in a route through the surgical map that is impossible. This is determined based on procedure specific rules (e.g., what phases must be present within the surgery) or based on the training data. This prevents any merges that result in an unrealistic surgical map.

The current merging method continues until either the maximum number of iterations has been reached, the score has converged, or there are no more possible merges. Determining the best tolerance of the score can be difficult as this can vary between datasets and procedures. In according with aspects an additional performance metric has been defined as the percentage of possible routes through the model that occur within the training data. This performance metric can identify whether too many merges have been carried out on the model, meaning that some information from the input data has been lost. The performance metric can be used instead of or in addition to the score of the model to identify when the merging process should be stopped.

In according to additional aspects, the method can be extended to include both surgical phase and surgical instrumentation data to provide a double layered model that shows the instrumentation model within each phase.

The extensions to the merging method described herein have several technical benefits. One technical benefit is an increase in speed and efficiency because the state values are applied based on distinct input observations rather than all observations individually, and/or due to multiple merges being allowed if they have the highest P(D|M) value and won't interact with each other. Another technical benefit is that the merging method is more robust because it prevents merges that would result in unrealistic paths through the model, and/or because it uses the performance metric based on the number of routes in the model that occur within the observation data to identify when merging should be stopped. Restricting merges can prevent the precision of the model from decreasing too much and preventing identification of multiple possible workflows that never occur in the data. Further technical benefits have to do with extending the merging method to include multiple layers of data (e.g., can have a model for surgical phase workflow with an instrumentation workflow model within each phase). This can allow users to see their overall workflow and break it down to look at each part in more detail.

Aspects described herein can be used to provide clinical insight by showing the most common workflows and the variations form this in a way that a user (e.g., a clinician or surgeon) can view the model and gain insights. In addition, outlier workflows in a dataset can be identified and flagged to the user. Further, workflow variation can be calculated and points within the workflow where there is the most variation can be identified which may turn out to be a decision point(s) for the surgeon.

Turning now to FIG. 15, a description and an illustration of a hidden Markov model (HMM) 1500 is generally shown in accordance with one or more aspects. In the example shown in FIG. 15 the observed values of a coin toss is heads (H) or tails (H), while the hidden values are whether the coin is fair (F) or biased (B). An HMM can be a statistical model where the system is assumed to be a Markov Process with unobservable or hidden state. Observed events can be used to learn about hidden states. A collection of Markov chain assumptions 1502 illustrates a plurality of states and observables. To predict the value of a state at time t+1, influences can include the value of the state at time t. Outcomes of an observable process can be influenced by outcomes of hidden states in a known way.

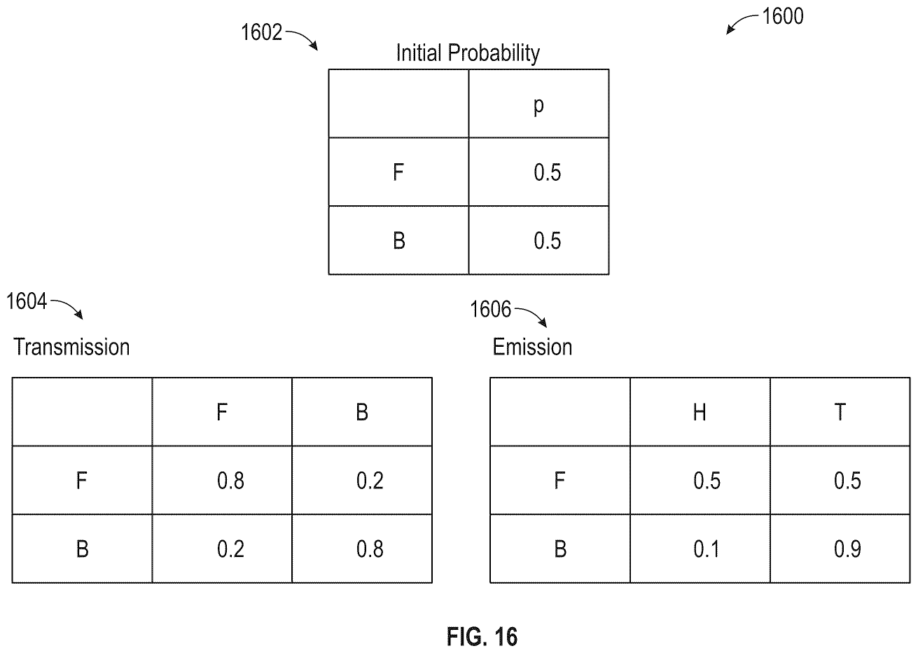

Turning now to FIG. 16, an illustration 1600 of Markov chain inputs is generally shown in accordance with one or more aspects. As shown in FIG. 16, a hidden Markov chain can be defined by three inputs: an initial probability 1602, a transmission matrix 1604, and an emission matrix 1606. As shown in FIG. 16, the initial probability is 50/50 for the coin being fair or biased. The initial probability 1602 can define a probability of a Gidden state being observed first in a sequence. The transmission matrix 1604 can include a probability of going from one hidden state to another. The emission matrix 1606 can include a probability of getting an observation from a hidden state.

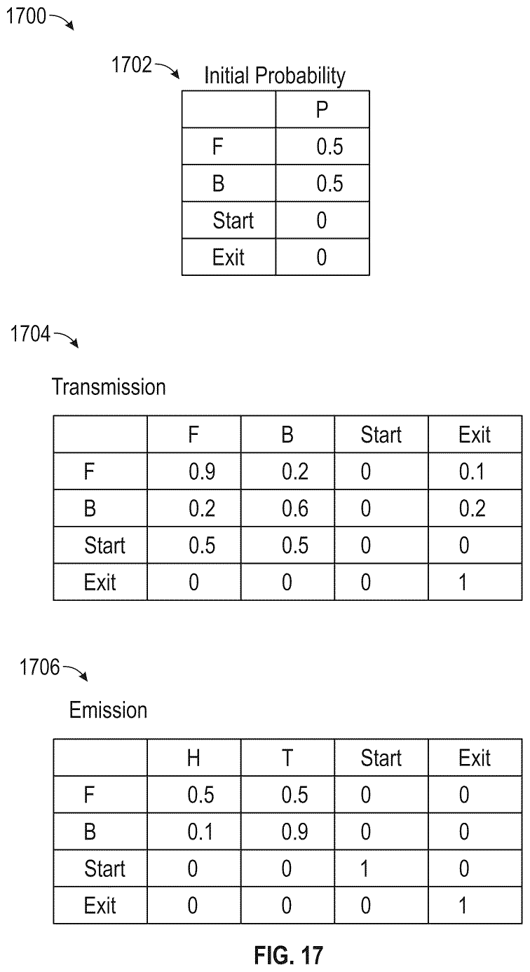

Turning now to FIG. 17, a description and an illustration of Markov chain inputs 1700 is generally shown in accordance with one or more aspects. Each of the inputs in FIG. 17 include two additional states: a start state and an exit state to model a process that has a starting point and an ending point for initial probability 1702, transmission matrix 1704, and emission matrix 1706. The initial probability 1702, transmission matrix 1704, and emission matrix 1706 expand upon the previously described initial probability 1602, transmission matrix 1604, and emission matrix 1606 of FIG. 16. Sequence data frames or sequences classes can be used to create the Markov chain inputs 1700, for example. In some aspects, the start row of the transmission matrix 1604 can be equivalent to the initial probability 1702.

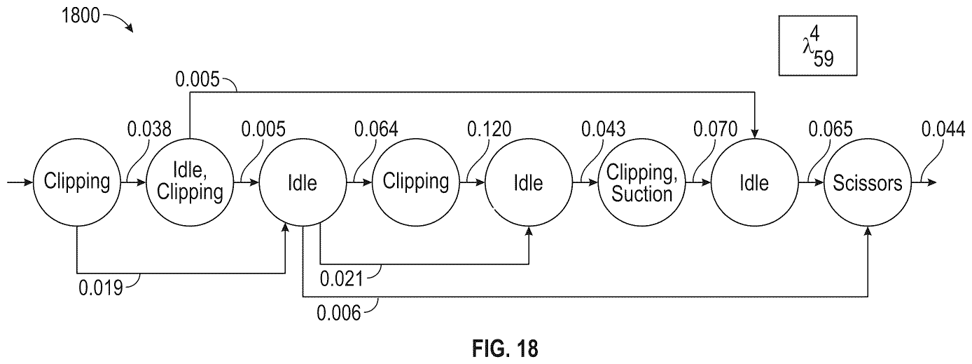

Turning now to FIG. 18, a visualization 1800 of a workflow is generally shown in accordance with one or more aspects. BMM can be used on surgical data to create the visualization 1800 shown in FIG. 18. For example, BMM can be used to create a model of surgical instrumentation with the aim to identify surgical phases, or to create a model of surgical phases and instrumentation with the aim to identify complications. BMM can be used to get the structure of the model and can result in an easy to read model of states from left to right (e.g., from initial model input to final model output) with no loops.

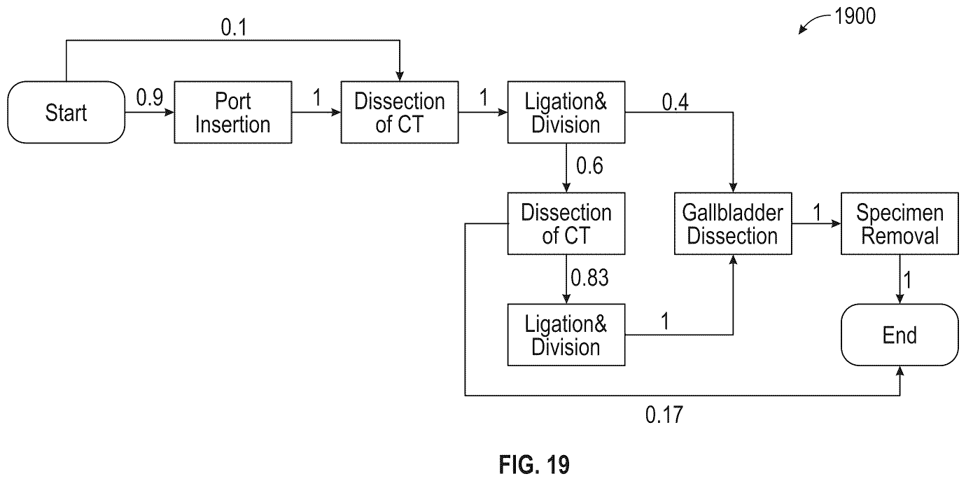

Turning now to FIG. 19, a visualization 1900 of a workflow is generally shown in accordance with one or more aspects. The visualization 1900 shown in FIG. 19 is a procedure map from start to finish, showing the different phases surgeons carry out and in what order. The visualization 1900 shown in FIG. 19 can be created using an HMM that has been modified to include a specified start and end to the process. This can be a helpful way to let surgeons view workflows for a procedure, as it depicts variation points and how often each variation happens. For example, port insertion may be the first phase 90% of the time after starting, while dissection may be the first phase 10% of the time after starting.

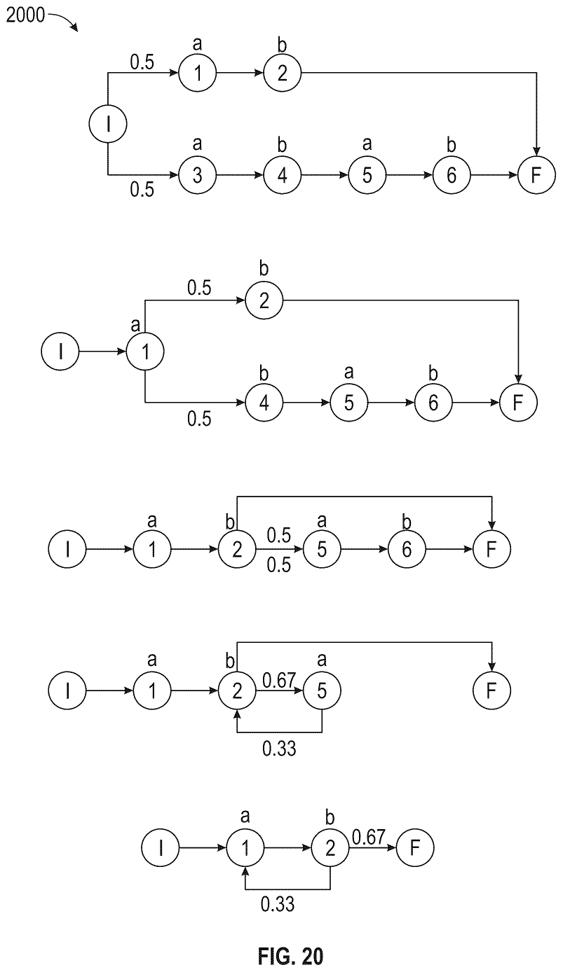

Turning now to FIG. 20, an example of BMM 2000 is generally shown in accordance with one or more aspects. Each node corresponds to a phase, or a step, in a medical procedure. Two sequences are shown in FIG. 20 between the initial node (I) and the ending node (F): ab and abab, where a and b are names of phases. Initially, each sequence can be made a branch on a model with separate sets of states. At each iteration, the process can find the best possible merge of two states, e.g., the one that increases the posterior probability of the training data. Possible merges do not include merges that will result in a loop in the model. The process can be repeated until reaching the maximum number of iterations or until the change in the posterior probability reaches tolerance. A goal of BMM 2000 is to optimize the probability of the input data D given the model structure M, e.g., optimize P(D|M).

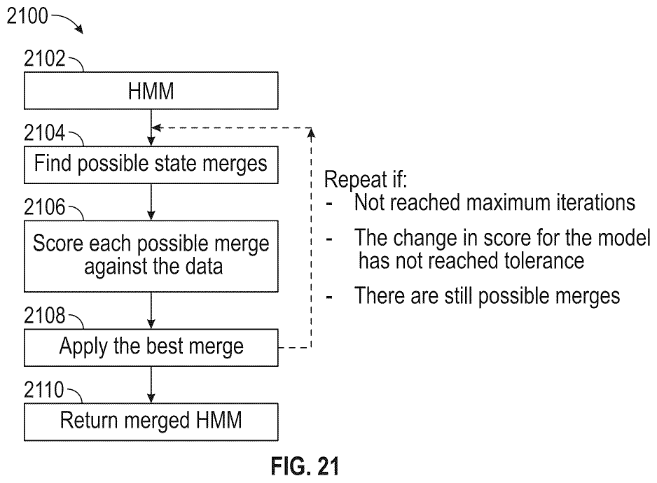

Turning now to FIG. 21, a flow diagram 2100 of a method for creating a merged HMM is generally shown in accordance with one or more aspects. At block 2102, an HMM is created using the input training data, with a branch for every workflow. This can include creating a transmission matrix, emission matrix and initial probability. At block 2104, possible state merges are found. A merge may be possible if both states result in the same observation and merging the states will not result in a loop in the model. At block 2106, each possible merge can be scored against given data. This can include applying the merge on a test HMM and updating the transmission matrix, with the score of the new model calculated for the given data. At block 2108, the best merge or merges can be applied. This can include finding a merge with the best scores, applying the merge to the model, and updating HMM parameters. Repeating blocks 2104, 2106, and 2108 can be performed until a maximum number of iterations is reached, the score has converged, and/or there are no more possible merges. At block 2110, the merged HMM is returned.

The processing shown in FIG. 21 is not intended to indicate that the operations are to be executed in any particular order or that all of the operations shown in FIG. 21 are to be included in every case. Additionally, the processing shown in FIG. 21 can include any suitable number of additional operations.

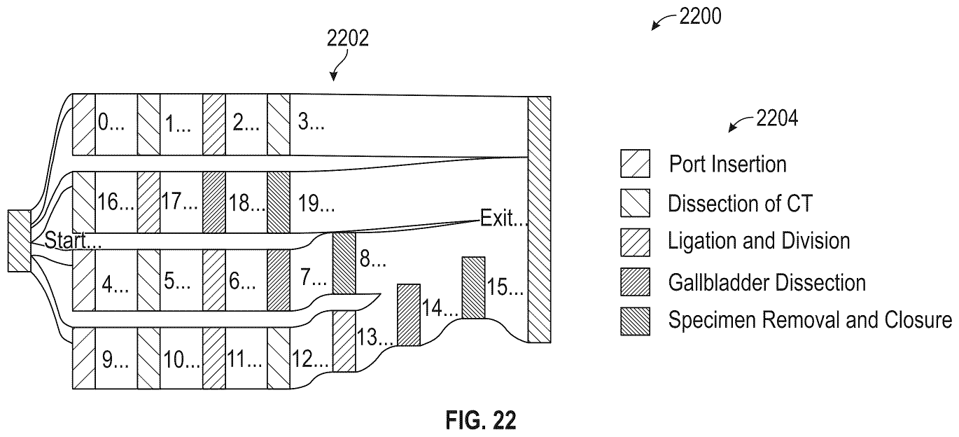

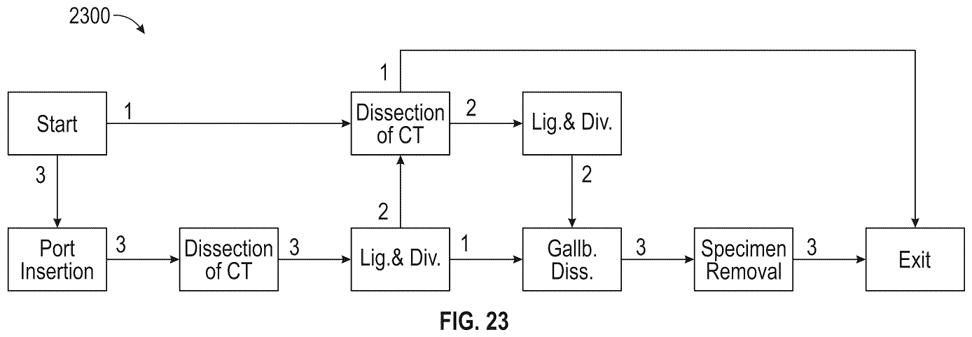

Turning now to FIG. 22, a visualization 2200 of an example of a model having four sequences is generally shown in accordance with one or more aspects. A sequence plot 2202 illustrates four sequences for a surgical procedure workflow, where each sequence has its own branch in the model with its own set of states. The states are then merged to create the merged model 2300 of FIG. 23. A legend 2204 can used to interpret the contents of the sequence plot 2202 as phases of the surgical procedure workflow.

Turning now to FIG. 23, a merged model 2300 of the example illustrated in FIG. 22 is generally shown in accordance with one or more aspects. The merged model 2300 can be read from left to right and shows how likely it is for each transition between phases. For example, 75% of the time the Port Insertion phase is expected to be first, and 25% of the time Dissection of Calot's triangle is expected to be first. It is noted that the example of FIG. 23 is not complete and may include unrealistic possible routes, such as transitioning from start to Dissection of Calot's triangle to exit.

There can be changes to the merging method. To change speed and efficiency, states can be applied based on distinct workflows and multiple best merges may be allowed if the merges do not interact, for example. Merging restrictions can be based on procedure requirements. This can prevent merges that will create routes from start to finish that do not meet the requirements of a procedure, e.g., routes that do not have all necessary phases. Alternative scoring can be used to identify over merging. For instance, scoring can be based on how many of the possible routes through the model occur in the training data, which can be used to identify when to stop the merging process as the model may become too generalized.

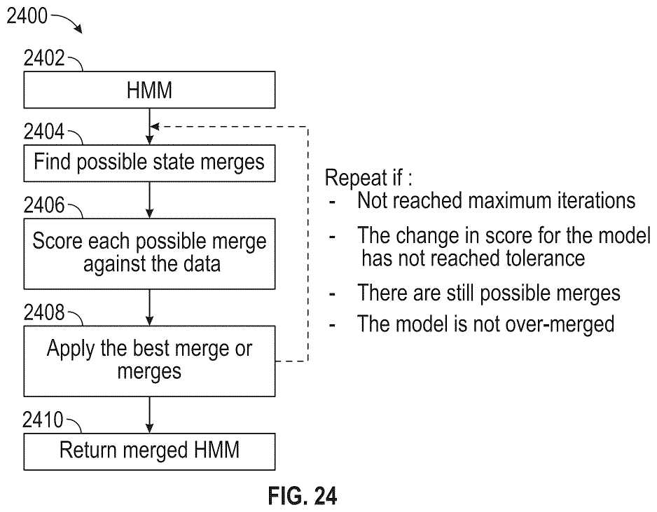

Turning now to FIG. 24, a flow diagram 2400 of an updated method for creating a merged HMM is generally shown in accordance with one or more aspects. At block 2402, an HMM can be created using the input training data, with a branch for every workflow. This can include creating a transmission matrix, emission matrix and initial probability. At block 2404, possible merge states are found. A merge may be possible if both states result in the same observation, merging the states will not result in a loop in the model, and merging the states will not result in an unrealistic path through the model. At block 2406, each possible merge can be scored against given data. This can include applying the merge on a test HMM and updating the transmission matrix, with the score of the new model calculated for the given data. At block 2408, the best merge or merges can be applied. This can include finding a merge with the best scores and applying the merge to the model. If there are other merges identified as best merges that do not interact with any previous merges, the additional best merges can also be applied. HMM parameters are updated with merges. Repeating blocks 2104, 2106, and 2108 can be performed until a maximum number of iterations is reached, the score has converged, there are no more possible merges, and/or an over-merged condition is detected. At block 2410, the merged HMM is returned.

The processing shown in FIG. 24 is not intended to indicate that the operations are to be executed in any particular order or that all of the operations shown in FIG. 24 are to be included in every case. Additionally, the processing shown in FIG. 24 can include any suitable number of additional operations.

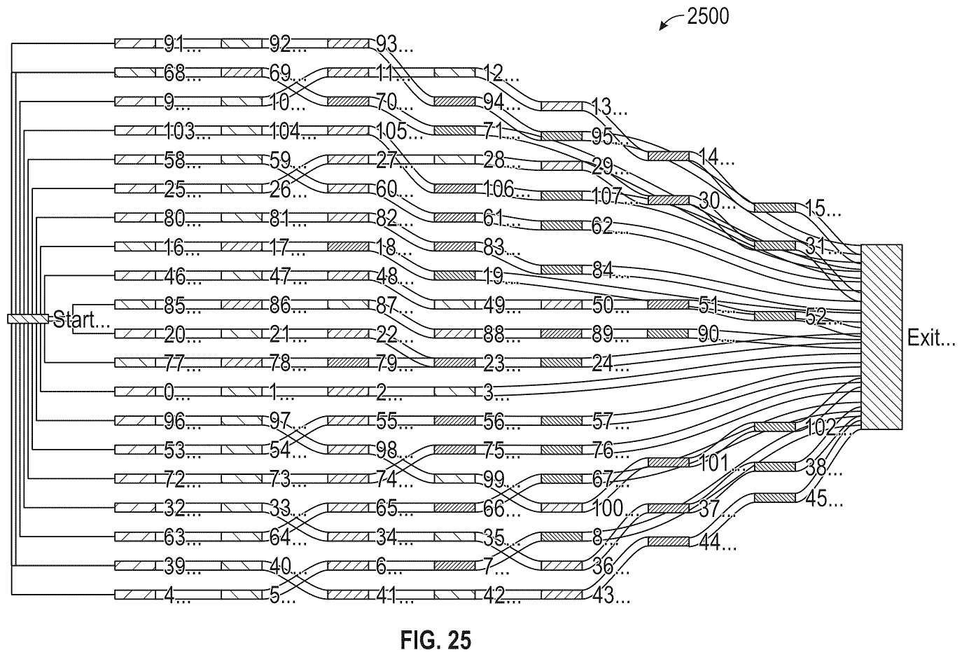

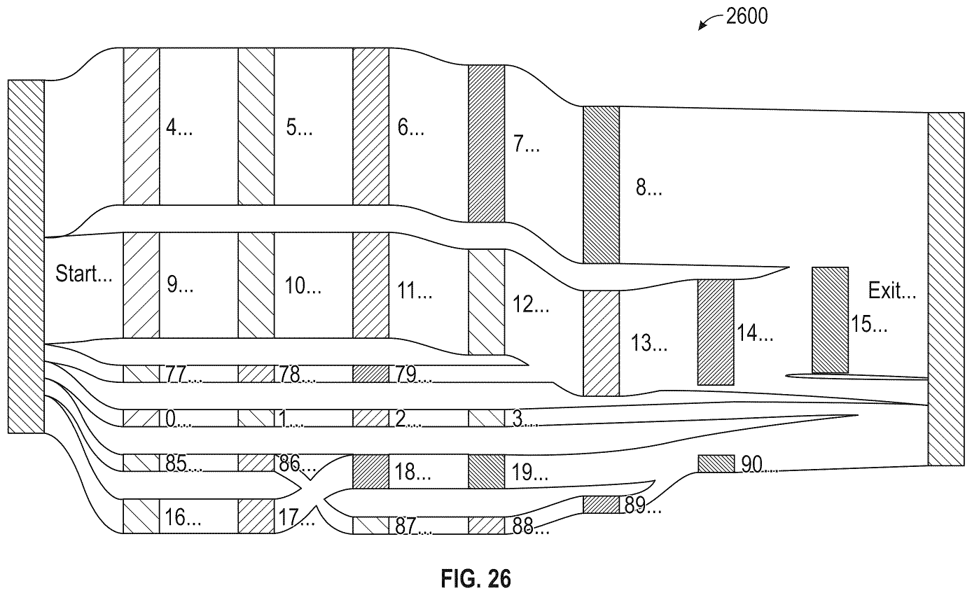

Turning now to FIG. 25, a visualization 2500 of states based on distinct workflows is generally shown in accordance with one or more aspects. As the training data increases in size, the time needed for the model merging increases with the number of states, and therefore possible merges increases. According to an aspect, to try to increase efficiency, the states of the unmerged model can be allocated based on distinct workflows. In the example of FIG. 25, in a surgical procedure with 20 workflows, with each workflow having its own set of states may take about 90 iterations until fully merged. This leads to a more complex and resource intensive process as compared to the example of FIG. 26.

Turning now to FIG. 26, a visualization 2600 of allowing multiple merges is generally shown in accordance with one or more aspects. The visualization 2600 uses the same 20 workflows as depicted in the visualization 2500 of FIG. 25. Rather than each workflow having its own set of states, in the example of FIG. 26, each distinct workflow has its own set of states, which can result in about 20 iterations until fully merged. This approach reduces the complexity and resource utilization as compared to the approach associated with FIG. 25.

Is some aspects, a single merge can be applied in every iteration. Initially, there may be multiple best merges. For instance, merging states 4 and 9 may be just as good as merging states 2 and 17. Allowing multiple merges can speed up the number of iterations needed. Multiple merges may be allowed if the merges do not interact with each other. For instance, merges can be allowed where there is no way through the model from a first merge to a second merge. For instance, merging of states 5 and 10 could conflict with merging states 4 and 9 since states 4 and 5 are in a sequence, and states 9 and 10 are in a sequence.

An example model when an additional merge constraint is used is generally shown in accordance with one or more aspects referring back to FIG. 23. Additional merge constraints can include restricting merges based on whether after the merge there is an unrealistic path through the model. For example, realistic/unrealistic paths can be defined based on procedural rules, such as all routes must include these phases, must start with port insertion, and must end with specimen removal. This can prevent merges such as one where there is an unrealistic route through the model, as previously described with respect to FIG. 23.

Following is an example use case of applying aspects of the pipeline described herein to publicly available datasets.

The proposed pipeline has been evaluated on two publicly released datasets: Cholec80 which contains 80 videos of cholecystectomy surgeries performed by 13 surgeons at University Hospital of Strasbourg, France, with annotations for surgical phases; and CATARACTS 2020 which contains 50 cataract surgeries performed at Brest University Hospital in 2015 by three surgeons. The surgical steps have been annotated by non-medical experts.

### Pipeline

Step 1—Identify rare phases. The first step of the pipeline is to identify atypical workflows based on frequency of the phases across the dataset. In one of the datasets, CATARACTS, there are rare phases which occur in less than 10% of the data. All cases with at least one rare phase were identified as atypical and excluded from further processing.

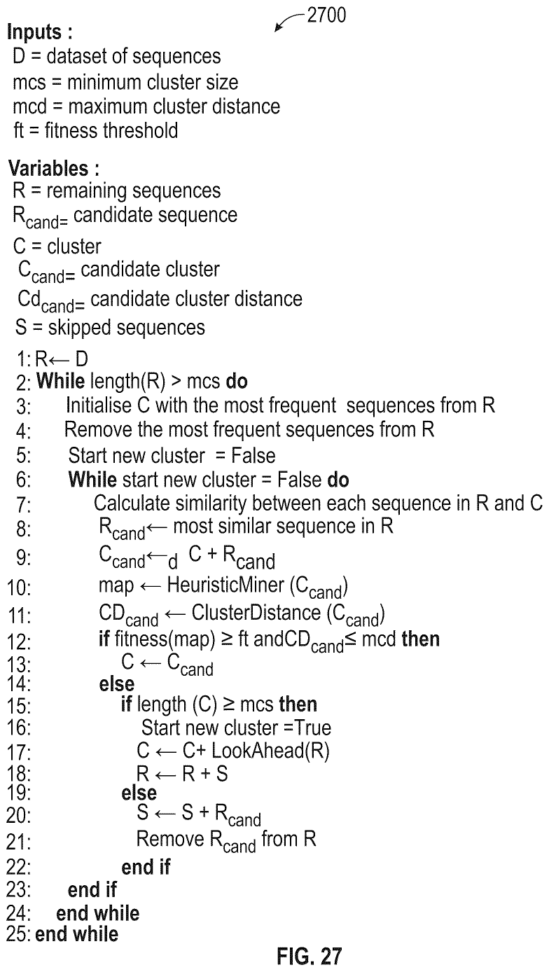

Step 2—Modified trace clustering. The next stage of the pipeline involves clustering the remaining cases using iterative model-based clustering. In accordance with aspects, an active trace clustering (ActiTraC) algorithm is modified as shown in the pseudocode 2700 in FIG. 27. First, to conserve deterministic “super-phases” in each cluster, the maximal repeated alphabet is replaced with common substrings as a metric for picking candidate sequences. An additional cluster membership constraint is also added based on the maximal intra-cluster distance to preserve the homogeneity of each cluster before a final cluster optimization is carried out.

The start of a cluster is the most frequent sequence, and the similarity metric is used to select a candidate cluster member. If the candidate cluster, i.e., the concatenation of the current cluster and the candidate sequence, meets two criteria (1) process map fitness is 100% and (2) maximum intra-cluster distance is below a threshold, the candidate sequence is accepted. Otherwise, it will be removed. This process is repeated until a candidate sequence does not meet the requirements. After this, the Look Ahead function from ActiTraC is applied, and a new cluster is started if there are enough remaining sequences. The number of clusters does not need to be predefined and is instead the number of clusters is determined by the input data and the stopping conditions.

The similarity metric can be calculated by finding how many common substrings are shared by the current cluster and the candidate sequence. Each remaining sequence is transformed into a Boolean vector whose values correspond to the occurrence of the common substrings. The similarity is the average Euclidean distance between the feature vector of the remaining sequence and the feature vectors of all sequences in the current cluster. The remaining sequence with the smallest similarity can be selected as the candidate sequence.

Heuristic Miner (HM) can be used to find the process map for the candidate cluster and to calculate the fitness for acceptance criteria. Moreover, in the case of multiple potential candidate sequences based on the similarity metric, the candidate sequence that results in the least complex model may be preferred.

After ActiTraC, a further cluster optimization process similar to a model-based-k-means clustering can be applied. Using leave-one-out cross validation, the most likely cluster can be found for each sequence based on the cluster process model. For each fold, the process models are calculated using HM, excluding the test sequence. This is repeated until there is convergence, resulting in each sequence being in the most applicable cluster. ActiTraC, with the additional constraint step introduced herein leads to tight initial clustering with respect to the feature vectors, while the process model-based optimization step may merge similar clusters. As such, aspects provide a hybrid approach that defines clusters based on both features and the likelihood of the process model.

Step 3—Create the process map. A modified form of the Bayesian Model Merging (BMM) method can be used to generate a flow diagram based on the clustered sequences. Unlike HM, BMM results in a process model free from loops. Surgical phase sequences, including those in the CATARACTS datasets, often have repeated phases. The visualization of the loop-free BMM process model can be read left-to-right and is therefore clearer and easier to interpret.

The original BMM method can be computationally expensive proportional to the square of the number of initial nodes. The following modifications are utilized by aspects to improve running efficiency. First, a common subsequence of consecutive phases is found in the dataset and merged into a single super-phase. This masking reduces the number of nodes and thus the number of possible merges in the BMM model. Additionally, greedy merging of nodes is replaced with beam search to allow multiple merges in one iteration for nodes that do not interact in the model. Finally, in the original BMM algorithm, the convergence threshold was set by information criteria. In accordance with aspects described herein, the number of iterations in the merging is determined by setting thresholds on the precision and complexity of the BMM model to ensure the model is understandable and represents the data.

After the masked model has converged, super-phases can be split back into the original phases and the model merging can be repeated. This allows the variations to re-merge with the components of the super-phases, simplifying the model visualization.

Step 4—Atypical case identification. The last step in actitrac is residual trace resolution; if no new clusters can be created, remaining sequences are assigned to a cluster based on process model likelihood. Rather than forcing the remaining sequences into a cluster, aspects forego this step and define remaining sequences as atypical.

Additionally, within each cluster, the likelihood of each if its members is calculated, given the cluster process model. It is assessed whether the likelihood is low by calculating the likelihoods of all possible sequences contained in the process model. Outlier bounds are found using the cumulative distribution function and any sequence with likelihood below that bound is identified as atypical.

Results. Cholec80. The videos in the Cholec80 dataset can be segmented into phase sequences using an alphabet of seven different phases. There may be six distinct phase sequences, the most common sequence constituting 64% of the data. “Preparation” is the only phase that does not appear in all cases, occurring in 89% of cases. Thus, for this dataset there were no rare phases.

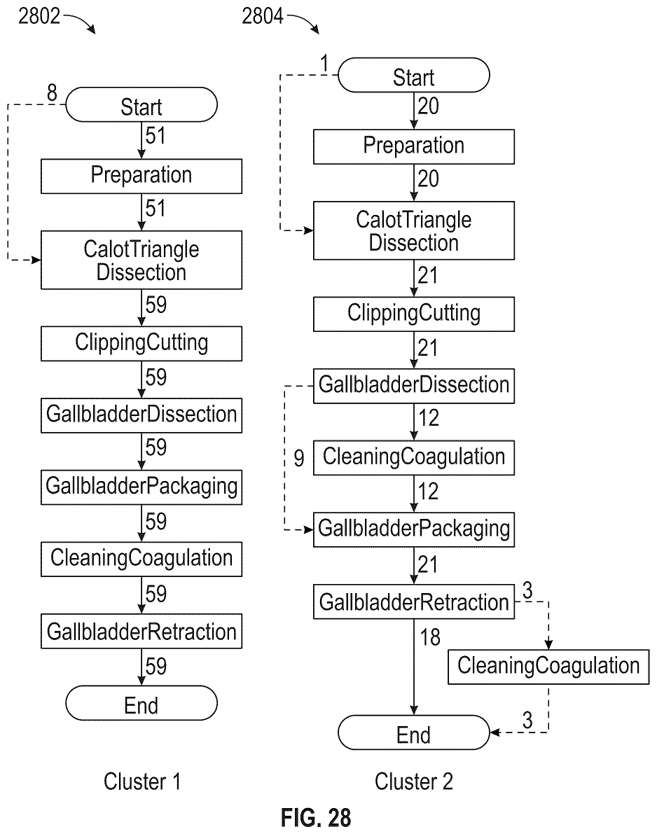

The dataset can be split into two clusters. The first cluster may contain the most common sequence and a minor variation of this workflow where the “Preparation” phase was missing. The second cluster can contain the rest of the data. Both clusters can have one substring that was common to at least 90% of the sequences in the cluster. BMM can be carried out on masked sequences, and a second round of model merging can be done after unmasking the super-phases. The ensuing process map visualizations of two clusters 2802 and 2804 are shown in FIG. 28. Each edge in the graph can be labelled with the number of sequences with that transition. For ease of visualization, infrequent edges can be shown as dotted lines and infrequent nodes as faded squares, allowing the frequent nodes and edges to stand out. For this dataset, no atypical cases were identified at any point of the pipeline.

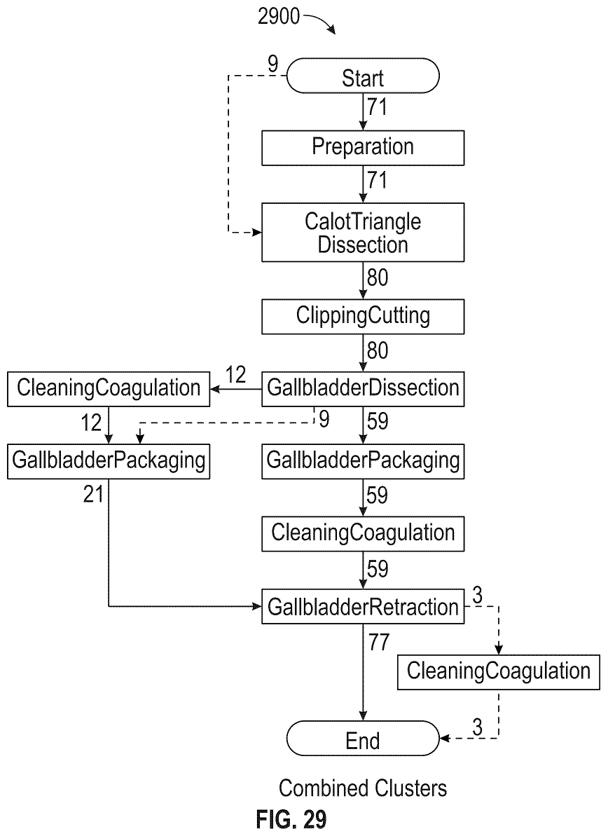

Due to the simplicity of the two process maps, and the similarities between them, a combined model could be generated to give an overall process model. For example, the clusters 2802 and 2804 can be combined as a process map 2900 of combined clusters in the visualization of FIG. 29. The combined process model (process map 2900) shows a clear pathway that most of the sequences follow. Minor variations, such as “CleaningCoagulation” occurring as the last phase of the sequence in three out of 80 videos, can be seen as faint deviations from the main path. A more common variation occurs after “GallbladderDissection”, which may indicate a point of decision for the operating surgeon to perform “CleaningCoagulation” before “GallbladderPackaging” rather than after it. These visual cues can help medical experts to focus on the most variable parts of the operation and do further clinical assessment.

As the amount of surgical data increases, so does the need for new methods of analysis to aid in its clinical interpretation. Aspects of the pipeline described herein can be used for different types and sizes of surgical datasets to identify a group of principal workflows and any atypical cases. This pipeline does not require any expert or procedure related inputs and can be run on any surgical data set. There is also room to extend the method to include more granular data including phase duration and instrument usage.

The use of modified ActiTraC can also allow the pipeline to deal with complex datasets where multiple clusters with idiosyncratic representative workflows were discovered. With the proposed modifications, the phase sequences can be clustered in a hybrid approach that is partly based on sequence features, namely common substrings within the data, and the quality of a process model of each cluster. In this way, coherent clusters can be found that highlight areas of consistent behavior and surgical variation for easier clinical analysis. Atypical phases and sequences can be identified and signposted by the pipeline for further clinical assessment.

Further, the use of common substrings in BMM can significantly increase efficiency, by reducing the number of initial nodes. Efficiency can also be improved by using beam search instead of greedy searching to identify the best merges. Motivated by the need for a simple visualization of the process model, stopping conditions can be modified to identify the best number of merges based on the precision and simplicity of the process model. Without this modification, the process models may be too complex to digest.

In accordance with aspects, the principal workflows can be clustered based on the common substrings within the data and are then visualized in an easy-to-understand process map. Additionally, any atypical cases can be identified as cases that may be of interest, this and the process map aid in the clinical interpretation of the data by simplifying. This has shown the benefit of the pipeline in carrying out initial analysis on a large data set in providing insights and visualizations of the data to facilitate clinical interpretation.

The use of active trace clustering allows the pipeline to be able to deal with more complex datasets, such as Gastric Bypass data, which contains multiple different approaches. This improves the ability of the pipeline to identify a wider range of principal workflows and more accurate atypical cases. The extensions to this method, including the use of the adjacency metric, the maximum cluster distance and use of the common substrings can be used to increase the differences between clusters and reduce the in-cluster distance.

In addition, the use of super-phases within the BMM significantly increases its efficiency, particularly for the more complex datasets of Cataract and Gastric Bypass data. The extensions to the BMM method also improve efficiency by allowing multiple merges in each iteration. They also were used to ensure the process map is robust, by allowing optional inputs on required phases during the operation and by identifying the best number of merges based on the precision and simplicity of the process map.

Aspects described herein can have applications within patient outcomes, with additional data such as complexity, complications and length of stay. Aspects of the pipeline and the resulting process maps can be used to understand the differences in workflow and their corresponding impact on patient outcomes. There is also room to extend the process models to include more granular data including phase duration and instrument usage.

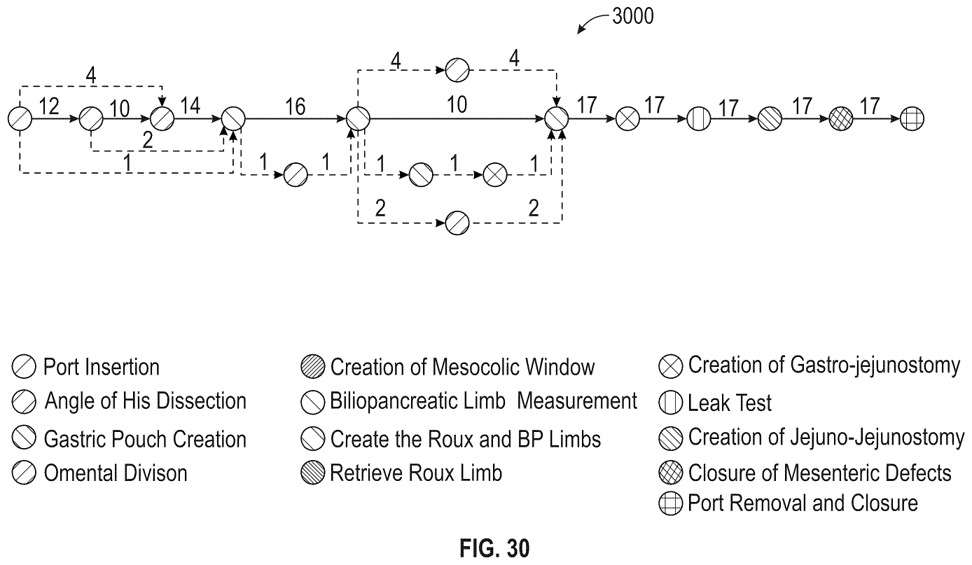

The visualizations 700, 1100, 1200, 1800, 1900, 2200, 2500, 2600, and 3000, clusters 2802, 2804, and process map 2900 shown in FIG. 7, FIG. 11, FIG. 12, FIG. 18, FIG. 19, FIG. 22, FIG. 25, FIG. 26, FIG. 28, FIG. 29, and FIG. 30 respectively, are examples of the type of data that may be generated and displayed to a user. In according to aspects, a graphical representation of the identified surgical approaches (or other data) is output for display on a display device. User input may be received via a user interface of the graphical representation and a second graphical representation including for example, providers that use one or more of the identified surgical approaches, may be output for display on the display device. In this manner, the visualization is interactive and can be modified based on input from a user. One skilled in the art will appreciate that any number of different visualizations containing different data and in different formats can be provided based on contents of the surgical workflow data for output to a display device, such as, for example, display 3119 of FIG. 31.

Turning now to FIG. 31, a computer system 3100 is generally shown in accordance with an aspect. The computer system 3100 can be an electronic computer framework comprising and/or employing any number and combination of computing devices and networks utilizing various communication technologies, as described herein. The computer system 3100 can be easily scalable, extensible, and modular, with the ability to change to different services or reconfigure some features independently of others. The computer system 3100 may be, for example, a server, desktop computer, laptop computer, tablet computer, or smartphone. In some examples, computer system 3100 may be a cloud computing node. Computer system 3100 may be described in the general context of computer-executable instructions, such as program modules, being executed by a computer system. Generally, program modules may include routines, programs, objects, components, logic, data structures, and so on that perform particular tasks or implement particular abstract data types. Computer system 3100 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 31, the computer system 3100 has one or more central processing units (CPU(s)) 3101a, 3101b, 3101c, etc. (collectively or generically referred to as processor(s) 3101). The processors 3101 can be a single-core processor, multi-core processor, computing cluster, or any number of other configurations. The processors 3101, also referred to as processing circuits, are coupled via a system bus 3102 to a system memory 3103 and various other components. The system memory 3103 can include one or more memory devices, such as read-only memory (ROM) 3104 and a random-access memory (RAM) 3105. The ROM 3104 is coupled to the system bus 3102 and may include a basic input/output system (BIOS), which controls certain basic functions of the computer system 3100. The RAM is read-write memory coupled to the system bus 3102 for use by the processors 3101. The system memory 3103 provides temporary memory space for operations of said instructions during operation. The system memory 3103 can include random access memory (RAM), read-only memory, flash memory, or any other suitable memory systems.

The computer system 3100 comprises an input/output (I/O) adapter 3106 and a communications adapter 3107 coupled to the system bus 3102. The I/O adapter 3106 may be a small computer system interface (SCSI) adapter that communicates with a hard disk 3108 and/or any other similar component. The I/O adapter 3106 and the hard disk 3108 are collectively referred to herein as a mass storage 3110.

Software 3111 for execution on the computer system 3100 may be stored in the mass storage 3110. The mass storage 3110 is an example of a tangible storage medium readable by the processors 3101, where the software 3111 is stored as instructions for execution by the processors 3101 to cause the computer system 3100 to operate, such as is described hereinbelow with respect to the various Figures. Examples of computer program product and the execution of such instruction is discussed herein in more detail. The communications adapter 3107 interconnects the system bus 3102 with a network 3112, which may be an outside network, enabling the computer system 3100 to communicate with other such systems. In one aspect, a portion of the system memory 3103 and the mass storage 3110 collectively store an operating system, which may be any appropriate operating system to coordinate the functions of the various components shown in FIG. 31.

Additional input/output devices are shown as connected to the system bus 3102 via a display adapter 3115 and an interface adapter 3116 and. In one aspect, the adapters 3106, 3107, 3115, and 3116 may be connected to one or more I/O buses that are connected to the system bus 3102 via an intermediate bus bridge (not shown). A display 3119 (e.g., a screen or a display monitor) is connected to the system bus 3102 by a display adapter 3115, which may include a graphics controller to improve the performance of graphics-intensive applications and a video controller. A keyboard, a mouse, a touchscreen, one or more buttons, a speaker, etc., can be interconnected to the system bus 3102 via the interface adapter 3116, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 31, the computer system 3100 includes processing capability in the form of the processors 3101, and storage capability including the system memory 3103 and the mass storage 3110, input means such as the buttons, touchscreen, and output capability including the speaker 3123 and the display 3119.

In some aspects, the communications adapter 3107 can transmit data using any suitable interface or protocol, such as the internet small computer system interface, among others. The network 3112 may be a cellular network, a radio network, a wide area network (WAN), a local area network (LAN), or the Internet, among others. An external computing device may connect to the computer system 3100 through the network 3112. In some examples, an external computing device may be an external web server or a cloud computing node.

It is to be understood that the block diagram of FIG. 31 is not intended to indicate that the computer system 3100 is to include all of the components shown in FIG. 31. Rather, the computer system 3100 can include any appropriate fewer or additional components not illustrated in FIG. 31 (e.g., additional memory components, embedded controllers, modules, additional network interfaces, etc.). Further, the aspects described herein with respect to computer system 3100 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects.

According to an aspect, a system includes a memory device and one or more processors coupled with the memory device. The one or more processors can be configured to access a surgical dataset that includes a plurality of phases associated with workflows of a surgical procedure, identify and remove cases with one or more rare phases, and cluster remaining cases without the one or more rare phases to form principal workflows and identify outliers. The one or more processors can further be configured to identify super-phases in the principal workflows, where the super-phases include subsequences of multiple phases. The one or more processors can further be configured to create a flow diagram that masks the super-phases from the principal workflows.

In some aspects, the phases can be determined based on video of multiple surgical cases of the surgical procedure. The one or more processors can be configured to identify one or more decision points in the flow diagram and characterize the outliers. The one or more processors can be configured to generate one or more models based on the principal workflows with the super-phases masked and perform a modified Bayesian model merging on the one or more models to create the flow diagram that is free of loops, where the modified Bayesian model merging can operate on the super-phases to reduce a number of nodes to merge.

In some aspects, the one or more processors can be configured to perform hidden Markov model merging on a model of the surgical dataset to generate a surgical workflow map. The hidden Markov model merging can include at least one of: allocating states of an unmerged model based on distinct workflows in the surgical dataset, performing multiple merges in a same iteration if the states do not interact, restricting merges based on whether after the merge, there is an unrealistic path through the model, and identifying when to stop merging based on how often a route occurs in training data.

In some aspects, the one or more processors can be configured to identify frequent sequences of the phases based on an input threshold value, remove a most variable phase from the infrequent sequences based on conditional entropy, find one or more most likely paths from a transition matrix based on the infrequent sequences, and identify effective infrequent sequences as most likely paths that have a similarity above the input threshold value when compared to other paths.

In some aspects, the one or more processors can be configured to select an input threshold value based on a distribution of sequences of phases, use higher-order transition matrices to create simulated paths based on determining that repeats of phases exist in the sequences, perform outlier identification on similarity scores that are based on adjacency, and identify effective infrequent sequences based on determining which sequences are not identified as the outliers.

In some aspects, the one or more processors can be configured to determine a probability of transitioning to each phase next to create a plurality of simulated paths, remove a simulated path based on the simulated path reaching a length restriction, find one or more most likely paths from a transition matrix based on the infrequent sequences, and identify effective infrequent sequences as most likely paths that have a similarity above the input threshold value when compared to other paths.

In some aspects, the one or more processors can be configured to select an input threshold value based on a distribution of sequences of phases, use higher-order transition matrices to create simulated paths based on determining that repeats of phases exist in the sequences, perform outlier identification on similarity scores that are based on adjacency, and identify effective infrequent sequences based on determining which sequences are not identified as the outliers.

In some aspects, the one or more processors can be configured to determine a probability of transitioning to each phase next to create a plurality of simulated paths, remove a simulated path based on the simulated path reaching a length restriction, compare each infrequent workflow to the simulated paths to create a similarity score, and form clusters based on the similarity score.

In some aspects, the one or more processors can be configured to determine upper bounds for outlier detection using a cumulative distribution function and classify sequences as effective infrequent sequences based on determining that the sequences are not identified as the outliers.

In some aspects, the one or more processors can be configured to identify a most common sequence of phases to start a cluster, identify a most similar sequence to the cluster to use as a test sequence, add the test sequence to the cluster based on determining that a new process map has a fitness above a threshold, add a sequence to the cluster based on determining that the sequence fits within the new process map, and begin a new cluster with one or more remaining sequences based on one or more requirements being met for the new cluster.

According to another aspect, a computer-implemented method for extracting principal workflows from a set of surgical phase sequences can include receiving the set of surgical phase sequences; identifying rare phases in the set of surgical phase sequences; removing workflows containing the rare phases from the set of surgical phase sequences; clustering the workflows in the set of surgical phase sequences using iterative model-based clustering; generating a flow diagram based on the clustered surgical phase sequences; and/or outputting the flow diagram via a user interface.

In some aspects, the flow diagram can include an indication of surgical decision points. Some aspects can include where prior to the clustering, at least a subset of the surgical phases is grouped together into super-phases that are treated by the clustering as a single surgical phase. Generating can include performing hidden Markov model merging on the clustered surgical phase sequences.

According to a further aspect, a computer program product includes a memory device with computer readable instructions stored thereon, where executing the computer readable instructions by one or more processing units causes the one or more processing units to perform a plurality of operations. The operations can include accessing a set of surgical phase sequences comprising a plurality of phases associated with workflows of a surgical procedure, identifying rare phases in the set of surgical phase sequences, removing workflows containing the rare phases from the set of surgical phase sequences, clustering the workflows in the set of surgical phase sequences, generating a flow diagram based on the clustered surgical phase sequences, and outputting the flow diagram via a user interface.

In some aspects, generating can include performing merging on the clustered surgical phase sequences.

In some aspects, merging can include creating a hidden Markov model using input training data with a transmission matrix, an emission matrix, and an initial probability, finding possible merges of states that result in a same observation and does not result in a loop, scoring each of the possible merges, applying a best merge of the hidden Markov model based on the scoring as a merged hidden Markov model, and providing the merged hidden Markov model to generate the flow diagram.

In some aspects, merging can include creating a hidden Markov model using input training data, finding possible merges of states that result in a same observation, does not result in a loop, and results in a realistic path through the hidden Markov model. The merging can also include scoring each of the possible merges, applying a best merge of the hidden Markov model based on the scoring as a merged hidden Markov model and an absence of interaction with a previous merge, and providing the merged hidden Markov model to generate the flow diagram.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1. A system comprising:
a memory device; and
one or more processors coupled with the memory device, the one or more processors configured to:
access a surgical dataset that includes a plurality of phases associated with workflows of a surgical procedure;
identify and remove cases with one or more rare phases;
cluster remaining cases without the one or more rare phases to form principal workflows and identify outliers;
identify super-phases in the principal workflows, wherein the super-phases comprise subsequences of multiple phases; and
create a flow diagram that masks the super-phases from the principal workflows.

2. The system of claim 1, wherein the phases are determined based on video of multiple surgical cases of the surgical procedure.

3. The system of claim 1, wherein the one or more processors are configured to identify one or more decision points in the flow diagram and characterize the outliers.

4. The system of claim 1, wherein the one or more processors are configured to:
generate one or more models based on the principal workflows with the super-phases masked; and
perform a modified Bayesian model merging on the one or more models to create the flow diagram that is free of loops, wherein the modified Bayesian model merging operates on the super-phases to reduce a number of nodes to merge.

5. The system of claim 4, wherein the modified Bayesian model merging is performed for a number of iterations in merging by setting thresholds on precision and complexity of the one or more models.

6. The system of claim 1, wherein the one or more processors are configured to:
perform modified Bayesian model merging on a model of the surgical dataset to generate a surgical workflow map.

7. The system of claim 6, wherein the modified Bayesian model merging comprises at least one of:
allocating states of an unmerged model based on distinct workflows in the surgical dataset;
performing multiple merges in a same iteration if the states do not interact;
restricting merges based on whether after the merge, there is an unrealistic path through the model; and
identifying when to stop merging based on how often a route occurs in training data.

8. The system of claim 1, wherein the one or more processors are configured to:
identify frequent sequences of the phases based on an input threshold value;
remove a most variable phase from the infrequent sequences based on conditional entropy;
find one or more most likely paths from a transition matrix based on the infrequent sequences; and
identify effective infrequent sequences as most likely paths that have a similarity above the input threshold value when compared to other paths.

9. The system of claim 1, wherein the one or more processors are configured to:
select an input threshold value based on a distribution of sequences of phases;
use higher-order transition matrices to create simulated paths based on determining that repeats of phases exist in the sequences;
perform outlier identification on similarity scores that are based on adjacency; and
identify effective infrequent sequences based on determining which sequences are not identified as the outliers.

10. The system of claim 1, wherein the one or more processors are configured to:
determine a probability of transitioning to each phase next to create a plurality of simulated paths;
remove a simulated path based on the simulated path reaching a length restriction;
compare each infrequent workflow to the simulated paths to create a similarity score; and
form clusters based on the similarity score.

11. The system of claim 10, wherein the one or more processors are configured to:
determine upper bounds for outlier detection using a cumulative distribution function; and
classify sequences as effective infrequent sequences based on determining that the sequences are not identified as the outliers.

12. The system of claim 1, wherein the one or more processors are configured to:
identify a most common sequence of phases to start a cluster;
identify a most similar sequence to the cluster to use as a test sequence;
add the test sequence to the cluster based on determining that a new process map has a fitness above a threshold;
add a sequence to the cluster based on determining that the sequence fits within the new process map; and
begin a new cluster with one or more remaining sequences based on one or more requirements being met for the new cluster.

13. A computer-implemented method for extracting principal workflows from a set of surgical phase sequences, the method comprising:
receiving the set of surgical phase sequences;
identifying rare phases in the set of surgical phase sequences;
removing workflows containing the rare phases from the set of surgical phase sequences;
clustering the workflows in the set of surgical phase sequences using iterative model-based clustering;
generating a flow diagram based on the clustered surgical phase sequences; and
outputting the flow diagram via a user interface.

14. The computer-implemented method of claim 13, wherein the flow diagram includes an indication of surgical decision points.

15. The computer-implemented method of claim 13, wherein prior to the clustering at least a subset of the surgical phases is grouped together into super-phases that are treated by the clustering as a single surgical phase.

16. The computer-implemented method of claim 13, wherein generating comprises performing modified Bayesian model merging on the clustered surgical phase sequences.

17. A computer program product comprising a memory device with computer readable instructions stored thereon, wherein executing the computer readable instructions by one or more processing units causes the one or more processing units to perform a plurality of operations comprising:
accessing a set of surgical phase sequences comprising a plurality of phases associated with workflows of a surgical procedure;
identifying rare phases in the set of surgical phase sequences;
removing workflows containing the rare phases from the set of surgical phase sequences;
clustering the workflows in the set of surgical phase sequences;
generating a flow diagram based on the clustered surgical phase sequences; and
outputting the flow diagram via a user interface.

18. The computer program product of claim 17, wherein generating comprises performing merging on the clustered surgical phase sequences.

19. The computer program product of claim 18, wherein merging comprises:
creating a hidden Markov model using input training data with a transmission matrix, an emission matrix, and an initial probability;
finding possible merges of states that result in a same observation and does not result in a loop;
scoring each of the possible merges;
applying a best merge of the hidden Markov model based on the scoring as a merged hidden Markov model; and
providing the merged hidden Markov model to generate the flow diagram.

20. The computer program product of claim 18, wherein merging comprises:
creating a hidden Markov model using input training data;
finding possible merges of states that result in a same observation, does not result in a loop, and results in a realistic path through the hidden Markov model;
scoring each of the possible merges;
applying a best merge of the hidden Markov model based on the scoring as a merged hidden Markov model and an absence of interaction with a previous merge; and
providing the merged hidden Markov model to generate the flow diagram.
